(19) United States
(12) Patent Application Publication
(10) Pub. No.: US 2017/0050989 A1
(43) Pub. Date: Feb. 23, 2017

(54) Molecular Imaging Probes

(71) Applicant: THE GENERAL HOSPITAL CORPORATION, Boston, MA (US)

(72) Inventor: Peter Caravan, Cambridge, MA (US)

(21) Appl. No.: 15/100,692

(22) PCT Filed: Dec. 3, 2014
(86) PCT No.: PCT/US2014/068444

§ 371 Date: Oct. 6, 2016

Related U.S. Application Data
(60) Provisional application No. 61/911,413, filed on Dec. 3, 2013.

(51) Int. Cl. C07F 5/00; G01N 33/68; C07F 1/08; G01N 33/574; A61K 49/10; A61K 51/04

(52) U.S. Cl. CPC C07F 5/003; A61K 49/106; A61K 51/0482; C07F 1/08; G01N 33/57488; G01N 33/6893; G01N 2333/765

(57) ABSTRACT

This disclosure relates to compounds of formula (I) shown below: [formula (I)], or a pharmaceutically acceptable salt thereof. These compounds can be used as imaging probes, e.g., for diagnosis of fibrosis or fibrogenesis.

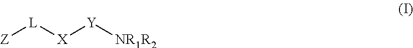

### CROSS-REFERENCE TO RELATED APPLICATIONS

This application claims the benefit of U.S. Provisional Application No. 61/911,413, filed Dec. 3, 2013, the entire disclosure of which is incorporated herein by reference.

### TECHNICAL FIELD

This disclosure relates to compounds that can be used as molecular imaging probes, as well as methods of making and using these compounds.

### BACKGROUND

Fibrosis is a ubiquitous reactive response to tissue injury. Scar tissue as a result of wound healing is a positive example of fibrosis. However in chronic tissue injury, ongoing cycles of injury and repair lead to accumulation of scar tissue and disruption of normal tissue architecture and function, which ultimately can result in organ failure. The cellular and molecular biology of fibrosis is similar whether it occurs in kidney, liver, lung or elsewhere and whether its cause is viral, chemical, physical or inflammatory. Fibrosis results from the excessive activity of fibroblasts and involves upregulation of a number of extracellular matrix proteins, such as type I collagen. Many therapeutic interventions can reverse fibrosis if detected early, however current radiological techniques only detect later stage disease where tissue damage may be irreversible.

### SUMMARY

This disclosure is based on the unexpected discovery that certain compounds containing an image group and a functional group that can react with an aldehyde group on collagen or elastin to attach (e.g., through a covalent bond) the compound to the collagen can be used as a molecular imaging probe (e.g., a magnetic resonance (MR) imaging probe) for diagnosis of disorders (e.g., fibrosis, fibrogenesis, atherosclerosis, myocardial infarct, or cancer).

In one aspect, this disclosure features a compound of formula (I):

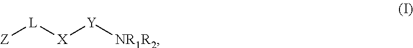

or a pharmaceutically acceptable salt thereof,
wherein X is —C(RₐR_{b})—, —C(S)—, or —C(O)—, in which each of Rₐ and R_{b}, independently, is H, alkyl, alkenyl, alkynyl, cycloalkyl, heteroaryl, or aryl; Y is —N(RC)— or —O—, in which R_{c} is H, alkyl, alkenyl, alkynyl, or aryl; L is —(CR_{d}Rₑ)ₙ—, —NH(CR_{f}R_{g})ₙ—, or —(CRₕRᵢ)ₙ-aryl-, in which each of R_{d}, Rₑ, R_{f}, R_{g}, Rₕ, and Rᵢ is independently in each instance H, alkyl, alkenyl, or alkynyl, and n is 1, 2, or 3; Z is a chelate group comprising a metal ion and a first complexing group, the first complexing group forming a metal complex with the metal ion; and each of R₁ and R₂, independently, is H or C₁-C₁₀ alkyl.

In another aspect, this disclosure features a method that includes administering to a mammal the compound of formula (I) above; and acquiring an image of a tissue of the mammal after administration of the compound.

In some embodiments, the image is a positron emission tomography image.

In some embodiments, the image is a single photon emission computed l tomography image.

In some embodiments, the image is a magnetic resonance image.

In some embodiments, the image is a computed tomography image.

In some embodiments, the image is a planar scintigraphy image.

In some embodiments, the first complexing group is a DOTA, NOTA, DO3AX, DO3AP, DOTP, DO2A2P, NOTP, NO2AP, NO2PA, TETA, TE2P, TE2A, TE1A1P, CBTE2P, CBTE1A1P, SBTE2A, SBTE1A1P, DTTP, CHX-A″-DTPA, Desferal, HBED, PyDO3P, PyDO2AP, PyDO3A, DIAMSAR, EDTA, DTP A, CB-TE2A, SarAr, PCTA, pycup, DEDPA, OCTAPA, AAZTA, DOTAIa, CyPic3A, TRAP, NOPO, or CDTA moiety.

In some embodiments, the metal ion is Gd³⁺, Mn³⁺, Mn³⁺, Fe³⁺, Ce³⁺, Pr³⁺, Nd³⁺, Eu³⁺, EU²⁺, Tb³⁺, Dy³⁺, ER³⁺, Ho³⁺, Tm³⁺, Yb³⁺, Cr³⁺ or an ion of a radioisotope selected from the group consisting of ⁶⁷Ga, ⁶⁸Ga, Al-¹⁸F, ⁶⁴Cu, ¹¹¹In, ⁵²Mn, ⁸⁹Zr, ⁸⁶Y, ²⁰¹TI, ^{94m}Tc, and ^{99m}Tc.

In some embodiments, Y is —N(R_{c})— or —O—, in which Rc is H, C₁-C₁₀ alkyl, C₂-C₁₀ alkenyl, C₂-C₁₀ alkynyl, or aryl. In some embodiments, Y is —NH— or —O—.

2

In some embodiments, X is —C(RₐR_{b})—, —C(S)—, or —C(O)—, in which each of Rₐ and R_{b}, independently, is H, C₁-C₁₀ alkyl, C₂-C₁₀ alkenyl, C₂-C₁₀ alkynyl, or aryl. In some embodiments, X is —CH₂— or —O—.

In some embodiments, L is wherein L is —(CH₂)ₙ—, —NH(CH₂)ₙ—, or —(CH₂)ₙ—aryl-, in which n is 1, 2, or 3. In some embodiments, L is —CH₂CH₂—, —NHCH₂—, —CH₂—Ph—, or —CH₂CH₂CH₂—.

In some embodiments, each of Rₐ and R_{b}, independently, is H or CH₃.

In some embodiments, Z further comprises a water molecule complexed with the metal ion.

In some embodiments, the tissue is selected from the group consisting of breast tissue, colon tissue, bone tissue, lung tissue, bladder tissue, brain tissue, bronchial tissue, cervical tissue, colorectal tissue, endometrial tissue, ependymal tissue, eye tissue, gallbladder tissue, gastric tissue, gastrointestinal tissue, neck tissue, heart tissue, liver tissue, pancreatic tissue, kidney tissue, laryngeal tissue, lip or oral tissue, nasopharyngeal tissue, oropharyngeal tissue, ovarian tissue, thyroid tissue, penile tissue, pituitary tissue, prostate tissue, rectal tissue, renal tissue, salivary gland tissue, skin tissue, stomach tissue, testicular tissue, throat tissue, uterine tissue, vaginal tissue, and vulvar tissue.

In some embodiments, the mammal is a human.

In some embodiments, each of R₁ and R₂ is H.

Provided herein is a method for assessing lysyl oxidase activity in an extracellular matrix of a biological sample, comprising administering to the extracellular matrix an imaging agent comprising a —NR—NH₂ or —O—NH₂ group, wherein R is H, C₁-C₁₀ alkyl, C₂-C₁₀ alkenyl, C₂-C₁₀ alkynyl, or aryl; and acquiring an image of the extracellular matrix after administration of the imaging agent.

Provided herein is a method for assessing lysyl oxidase activity in a tissue or in a tumor in a mammal, comprising administering to the mammal an imaging agent comprising a—NR—NH₂ or —O—NH₂ group, wherein R is H, C₁-C₁₀ alkyl, C₂-C₁₀ alkenyl, C₂-C₁₀ alkynyl, or aryl; and acquiring an image after administration of the imaging agent.

Further provided herein is a method for imaging an extracellular matrix of a biological sample, a tissue in a mammal, or a tumor in a mammal, comprising administering to the extracellular matrix an imaging agent comprising a —NR—NH₂ or —O—NH₂ group, wherein R is H, C₁-C₁₀ alkyl, C₂-C₁₀ alkenyl, C₂-C₁₀ alkynyl, or aryl; and acquiring an image of the extracellular matrix after administration of the compound.

Further provided herein is a method for imaging a tissue or a tumor in a mammal, comprising administering to the mammal an imaging agent comprising a —NR—NH₂ or —O—NH₂ group, wherein R is H, C₁-C₁₀ alkyl, C₂-C₁₀ alkenyl, C₂-C₁₀ alkynyl, or aryl; and acquiring an image of the mammal after administration of the compound.

Further provided herein is a method for assessing the level of fibrosis in a tissue in a mammal, comprising administering to the mammal an imaging agent comprising a—NR—NH₂ or —O—NH₂ group, wherein R is H, C₁-C₁₀ alkyl, C₂-C₁₀ alkenyl, C₂-C₁₀ alkynyl, or aryl; and acquiring an image of the mammal after administration of the compound.

Further provided herein is a method for diagnosing a fibrotic disease in a mammal, comprising administering to the mammal an imaging agent comprising a —NR—NH₂ or —O—NH₂ group, wherein R is H, C₁-C₁₀ alkyl, C₂-C₁₀ alkenyl, C₂-C₁₀alkynyl, or aryl; and acquiring an image of the mammal after administration of the compound.

In some embodiments, the fibrotic disease is selected from the group consisting of: pulmonary fibrosis, chronic obstructive pulmonary disease, pulmonary arterial hypertension, heart failure, hypertrophic cardiomyopathy, myocardial infarction, atrial fibrillation, diabetic nephropathy, systemic lupus erythematosus, polycystic kidney disease, glomerulonephritis, end stage renal disease, nonalcoholic steatohepatitis, alcoholic steatohepatitis, hepatitis C virus infection, hepatitis B virus infection, primary sclerosing cholangitis, inflammatory bowel disease, scleroderma, atherosclerosis, glaucoma, diabetic retinopathy, radiation induced fibrosis, surgical adhesions, cystic fibrosis, and cancer. For example, the fibrotic disease can be idiopathic pulmonary fibrosis.

In some embodiments, the fibrotic disease is a cancer selected from the group consisting of: a breast cancer, a colon cancer, a bone cancer, a lung cancer, a bladder cancer, a brain cancer, a bronchial cancer, a cervical cancer, a colorectal cancer, an endometrial cancer, an ependymoma, a retinoblastoma, a gallbladder cancer, a gastric cancer, a gastrointestinal cancer, a glioma, a head and neck cancer, a heart cancer, a liver cancer, a pancreatic cancer, a melanoma, a kidney cancer, a laryngeal cancer, a lip or oral cancer, a mesothioma, a mouth cancer, a myeloma, a nasopharyngeal cancer, a neuroblastoma, an oropharyngeal cancer, an ovarian cancer, a thyroid cancer, a penile cancer, a pituitary cancer, a prostate cancer, a rectal cancer, a renal cancer, a salivary gland cancer, a sarcoma, a skin cancer, a stomach cancer, a testicular cancer, a throat cancer, a uterine cancer, a vaginal cancer, and a vulvar cancer.

In some embodiments, the imaging agent used in a method described herein is a compound of formula (I), or a pharmaceutically acceptable salt thereof.

In some embodiments, the method of the disclosure further comprises evaluating the signal level after administration of the imaging agent with the signal level of a control.

In some embodiments, the method of the disclosure further comprises determining whether the tumor is cancerous upon evaluating the signal level after administration of the imaging agent with the signal level of a control.

Other features, objects, and advantages will be apparent from the description, drawings and the claims.

### DESCRIPTION OF DRAWINGS

FIG. 1A are axial liver MR images (L=liver, S=stomach) of fibrotic mouse pre- and 30-minute post administration of probe Compound 1 (i.e., Gd-Hyd). The images show strong MR signal enhancement after administration of Compound 1.

FIG. 1B are axial liver MR images of fibrotic mouse pre- and 30-minute post administration of control probe Compound 2 (i.e., Gd-Me2-Hyd). The images show little MR signal enhancement of fibrotic liver.

FIG. 1C shows enhanced liver/muscle contrast in fibrotic mice that received probe Compound 1, but not in control mice that had healthy livers and received Compound 1 or in fibrotic mice that received control probe Compound 2.

FIG. 1D shows that Sirius Red staining confirms advanced fibrosis in fibrotic mice.

FIGS. 2A and 2B are coronal MR images of a sham mouse and a mouse with pulmonary fibrosis, respectively. False color overlay is the difference in image of 30-minute post administration of Compound 1 (0.1 mmol/kg) and the baseline image, which shows extensive enhancement of the fibrotic lung, but very little enhancement of the lungs of the sham mouse.

FIGS. 2C and 2D show images obtained pre- (left) and 2-minute post administration of Compound 1 (right) in sham mouse and fibrotic mouse, respectively. The images show strong and similar initial MR signal enhancement of the blood pool, demonstrating full injection of Compound 1 to both mice.

FIG. 2E shows change in lung/muscle contrast to noise ratio (CNR) after injection of Compound 1 (at 1 hour post injection). Change in CNR (Delta CNR) is highly elevated in fibrotic mice (p=0.0001).

FIG. 2F shows H&E staining (left) and Sirius Red staining (right) results of pulmonary fibrosis in the mouse treated with bleomycin (bottom panels) compared to the normal lungs of the sham mouse (top panels).

FIG. 3 depicts relaxivity characteristics of Compound 1 and Compound 2with unmodified bovine serum albumin (BSA) or modified bovine serum albumin (BSA-ALD). FIG. 3a shows relaxivity (mM⁻¹sec⁻¹) for each preparation. FIG. 3b shows % change in relaxivity (mM⁻¹sec⁻¹).

FIG. 4 shows levels of Gd bound to unmodified bovine serum albumin (BSA) or modified bovine serum albumin (BSA-ALD) in an in vitro binding assay. FIG. 4a depicts nmol Gd bound to protein in each preparation. FIG. 4b depicts % Gd bound to protein in each preparation.

FIG. 5A shows % change in relaxation time for unmodified bovine serum albumin (BSA) or modified bovine serum albumin (BSA-ALD) bound and free solution fraction after separation.

FIG. 5B shows T₁ relaxivity measurements for Compound 1 in modified bovine serum albumin (BSA-ALD) before and after separation.

FIG. 6 shows Compound 1 imaging of liver fibrosis progression in CCl₄-treated mice after 6 or 12 weeks. FIG. 6A shows a representative image of vehicle control mouse before (pre, left panel) and 15 minutes after Compound 1 injection (post, right). L=liver, S=stomach, M=muscle. Little enhancement between pre- and post images is seen in vehicle. FIG. 6B shows enhancement seen in the 6-week CC₄-treated mice. FIG. 6C shows enhancement seen in the 12-week CCl₄-treated mice.

FIG. 7 shows the quantification of Compound 1 imaging of liver fibrosis progression. ΔCNR increases from 0.1 in vehicle control group (veh, open bar) to 1.2 after 6 weeks of CCl₄ (16 w, 2-fold increase, grey bar), and further increases to 2.0 (20fold increase) by 12 weeks (12 w, black bar). **p<0.01, ****p<0.0001, ANOVA.

FIG. 8 shows histology and lysyl oxidase expression in mice. FIG. 8A: Sirius red staining shows portal fibrosis and occasional bridging in 6-week CCl₄-treated animals (6 wk). 12-week CCl₄-treated animals have complete bridging fibrosis (12 wk). Vehicle shows background staining (veh). FIG. 8B: Collagen content quantified by Sirius red staining shows 0.6% in vehicle, significantly increases to 2.7% in 6-week animals, and to 4.0% in 12-week CCl₄ liver. qRT-PCR of lysyl oxidase expression shows levels of LOX (FIG. 8C), LOXL2 (FIG. 8D), and LOXL1 (FIG. 8E) with CCl₄ treatment. In FIG. 8B, ***p<0.001, ****p<0.0001, ANOVA. In C-E, **p<0.01, ****p<0.0001, t-test.

FIG. 9 shows quantification of Compound 1 imaging of liver fibrosis regression. Mice that received CCl₄ for 6 weeks followed by a 6 week recovery period (6 w-r) show less liver signal enhancement by Gd-Hyd than mice that were imaged after 6 weeks (6 w) or 12 weeks (12 w) of CCl₄ treatment. While the 6w and 12w groups show ΔCNR significantly higher than the vehicle control group (veh), there is no significant difference between the 6w-r group and the veh group to 1.2 (6-week CCl₄), and reduces to 0.5 after 6-week of CCl₄ withdrawal. **p<0.01, ****p<0.0001, ns=not significant, ANOVA.

FIG. 10 shows Compound 1 imaging of disease progression in mice treated with bleomycin. Signal enhancement in the lung is shown here superimposed on the anatomical images. FIG. 10A: PBS-injected sham animals have little to no Compound 1 update. The uptake of Compound 1 increased in the 1-week bleomycin-treated animals (FIG. 10B), and further increased in the 2-week bleomycin treated animals (FIG. 10C).

FIG. 11 shows pathological measures that confirm disease severity of bleomycin-treated mice. FIG. 11A shows that bleomycin-induced fibrotic mice have an average Ashcroft score of 4.1 at 1 -week post bleo injection, 5.3 at 2-week post bleo, and 0 in the PBS sham. FIG. 11B: Area of positive Sirius red staining increases slightly in the 1-week bleo cohort (0.17%) compared to 0.09% in the PBS controls, and significantly increases to 0.30% in the 2-week bleo animals. FIG. 11C: The injury area defined by H&E staining is 0.3% in the sham, increases to 4.6% in 1-week bleo, and further increases to 15.0%. ***p<0.001, ****p<0.0001 ANOVA.

### DETAILED DESCRIPTION

In general, this disclosure relates to compounds that can be used as molecular imaging probes, as well as methods of making and using these compounds.

### Compounds

The term “alkyl” refers to a saturated, linear or branched hydrocarbon moiety, such as —CH₃ or —CH(CH₃)₂. The terms “alkenyl” and “alkynyl” refer to substituted or unsubstituted unsaturated aliphatic groups analogous to the alkyls described above, but that contain at least one double or triple bond, respectively. The term “aryl” refers to a hydrocarbon moiety having one or more aromatic rings. Examples of aryl moieties include phenyl (Ph), phenylene, naphthyl, naphthylene, pyrenyl, anthryl, and phenanthryl. Alkyl and aryl mentioned herein include both substituted and unsubstituted moieties, unless specified otherwise.

The term “heteroaryl” includes substituted or unsubstituted aromatic 5- to 7-membered ring structures, more preferably 5- to 6-membered rings, whose ring structures include one to four heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, isoxazole, oxazole, oxadiazole, thiazole, thiadiazole, triazole, pyrazole, pyridine, pyrazine, pyridazine and pyrimidine, and the like.

The terms “carbocycle” and “carbocyclyl”, as used herein, refer to a non-aromatic substituted or unsubstituted ring in which each atom of the ring is carbon. The terms “carbocycle” and “carbocyclyl” also include polycyclic ring systems having two or more cyclic rings in which two or more carbons are common to two adjoining rings wherein at least one of the rings is carbocyclic, e.g., the other cyclic rings can be cycloalkyls, cycloalkenyls, cycloalkynyls, aryls, heteroaryls, and/or heterocyclyls.

The term “cycloalkyl”, as used herein, refer to a saturated substituted or unsubstituted ring in which each atom of the ring is carbon.

The terms “cycloalkenyl” and “cycloalkynyl” as used herein refer to cycloalkyl groups that bear at least one double bond and triple bond, respectively, within the ring.

The term “heterocyclyl” or “heterocycloalkyl” refers to substituted or unsubstituted non-aromatic 3- to 10-membered ring structures, for example, 3- to 7-membered rings, whose ring structures include one to four heteroatoms. The ring may be completely saturated or may have one or more unsaturated bonds such that the ring remains non-aromatic. Heterocyclyl rings contain 1-2 atoms which are members of the group consisting of: NH, N, N(C₁₋₆allcyl), O, and S. The term “heterocyclyl” or “heterocycloalkyl” also includes polycyclic ring systems having two or more cyclic rings in which one or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Heterocyclyl groups may be optionally substituted with 1, 2, or 3 substituents each independently selected from the group consisting of halo, cyano, nitro, hydroxyl, C₁₋₆alkoxy, heteroallcyl, C₆₋₁₀ aryloxy, C₁₋₆aralkoxy, CF₃, quaternary ammonium ion, sugar, C₁₋₆ alkyl, —C(═O)(C₁₋₆ alkyl), —SO₂(C₁₋₆ alkyl), —C(═O)O(C₁₋₆ alkyl),—C(═O)O(heteroalkyl), —C(═O)NH(C₁₋₆ alkyl), —C(═O)NH(heteroalkyl), —C(═O)(phenyl), —SO₂(phenyl), and phosphate (or a salt thereof). Examples of polycyclic heterocyclyls include 6-azabicyclo[3.1.1]heptane, 3-oxa-6-azabicyclo[3.1.1]heptane, 5-azaspiro[2.4]heptane, 2-oxaspiro[3.3]heptane, octahydrobenzofuran, 1,2,3,4-tetrahydroquinoline, and octahydro-1H-quinolizine.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. Possible substituents on aryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀alkylsulfonamino, arylsulfonamino, C₁-C₁₀ alkylimino, arylimino, C₁-C₁₀alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl include all of the above-recited substituents except C₁-C₁₀ alkyl. Possible substituents on alkenyl include all of the above-recited substituents for aryl except C₂-C₁₀ alkenyl. Possible substituents on alkynyl include all of the above-recited substituents for aryl except C₂-C₁₀ alkynyl. Possible substituents on heteroaryl, heterocycloalkyl, and carbocyclyl include all of the above-recited substituents for aryl.

In some embodiments, this disclosure relates to the compounds of formula (I):

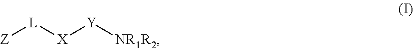

or a pharmaceutically acceptable salt thereof,
wherein X is —C(RₐR_{b})—, —C(S)—, or —C(O)—, in which each of Rₐ and R_{b}, independently, is H, alkyl, alkenyl, alkynyl, cycloalkyl, heteroaryl, or aryl; Y is —N(R_{c})— or —O—, in which Rc is H, alkyl, alkenyl, alkynyl, or aryl; L is —(CR_{d}Rₑ)ₙ—, —NH(CR_{f}R_{g})ₙ—, or —(CRₕRᵢ)ₙ-aryl-, in which each of R_{d}, Rₑ, R_{f}, R_{g}, Rₕ, and Rᵢ is independently in each instance H, alkyl, alkenyl, or alkynyl, and n is 1, 2, or 3; Z is a chelate group comprising a metal ion and a first complexing group, the first complexing group forming a metal complex with the metal ion; and each of R₁ and R₂, independently, is H or C₁-C₁₀ alkyl.

In some embodiments, each of R₁ and R₂ is H.

In some embodiments, a compound of formula (I) has the structure of formula (Ia):

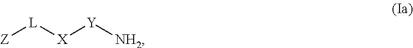

or a pharmaceutically acceptable salt thereof,
wherein X is —C(RₐR_{b})—, —C(S)—, or —C(O)—, in which each of Rₐ and R_{b}, independently, is H, alkyl, alkenyl, alkynyl, cycloalkyl, heteroaryl, or aryl; Y is —N(R_{c})— or —O—, in which Rc is H, alkyl, alkenyl, alkynyl, or aryl; L is —(CR_{d}Rₑ)ₙ—, —NH(CR_{f}R_{g})ₙ—, or —(CRₕRᵢ)ₙ-aryl-, in which each of R_{d}, Rₑ, R_{f}, R_{g}, Rₕ, and Rᵢ is independently in each instance H, alkyl, alkenyl, or alkynyl, and n is 1, 2, or 3; Z is a chelate group comprising a metal ion and a first complexing group, the first complexing group forming a metal complex with the metal ion.

In some embodiments, Y is —N(R_{c})— or —O—, in which Rc is H, C₁-C₁₀ alkyl, C₂-C₁₀ alkenyl, C₂-C₁₀ alkynyl, or aryl. In some embodiments, Y is —NH— or —O—.

In some embodiments, X is —C(RₐR_{b})—, —C(S)—, or —C(O)—, in which each of Rₐ and R_{b}, independently, is H, C₁-C₁₀ alkyl, C₂-C₁₀ alkenyl, C₂-C₁₀ alkynyl, or aryl. In some embodiments, X is —C(RₐR_{b})— or —C(O)—, in which each of Rₐ and R_{b}, independently, is H, C₁-C₁₀ alkyl, C₂-C₁₀ alkenyl, C₂-C₁₀ alkynyl, or aryl. In some embodiments, X is —CH₂— or —O—. For example, X can be —CH₂—.

In some embodiments, L is wherein L is —(CH₂)ₙ—, —NH(CH₂)ₙ—, or —(CH₂)ₙ—aryl-, in which n is 1, 2, or 3. In some embodiments, L is —CH₂CH₂—, —NHCH₂—, —CH₂—Ph—, or —CH₂CH₂CH₂—.

In some embodiments, each of Rₐ and R_{b}, independently, is H or CH₃.

In some embodiments, Z further comprises a water molecule complexed with the metal ion.

The first complexing group generally comprises nitrogen and/or carboxylate moieties that can bind to metal ions. Metal complexing groups are known in the art, for example, as described for Gd³⁺ complexes in Hermann, P. et al. Dalton Transactions 2008, 3027-3047, hereby incorporated by reference in its entirety. In some embodiments, the first complexing group is a DOTA, NOTA, DO3AX, DO3AP, DOTP, DO2A2P, NOTP, NO2AP, NO2PA, TETA, TE2P, TE2A, TE1A1P, CBTE2P, CBTE1A1P, SBTE2A, SBTE1A1P, DTTP, CHX-A″-DTPA, Dcsfcral, HBED, PyDO3P, PyDO2AP, PyDO3A, DIAMSAR, EDTA, DTP A, CB-TE2A, SarAr, PCTA, pycup, DEDPA, OCTAPA, AAZTA, DOTAIa, CyPic3A, TRAP, NOPO, or CDTA moiety. In some embodiments, the first complexing group is a DOTA, NOTA, EDTA, DTP A, CB-TE2A, SarAr, PCTA, pycup, or CDTA moiety. Examplary representations of the complexing group include the following, with possible points of attachment to the remainder of the molecule indicated with the wavy ( [symbol] ) lines:

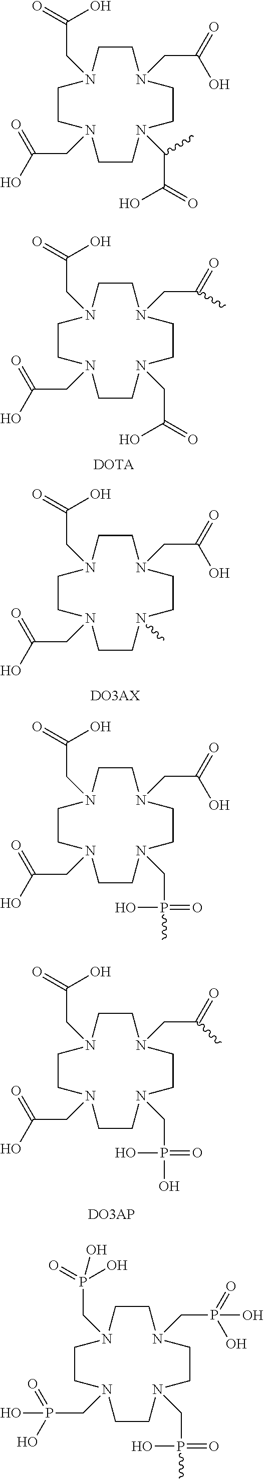

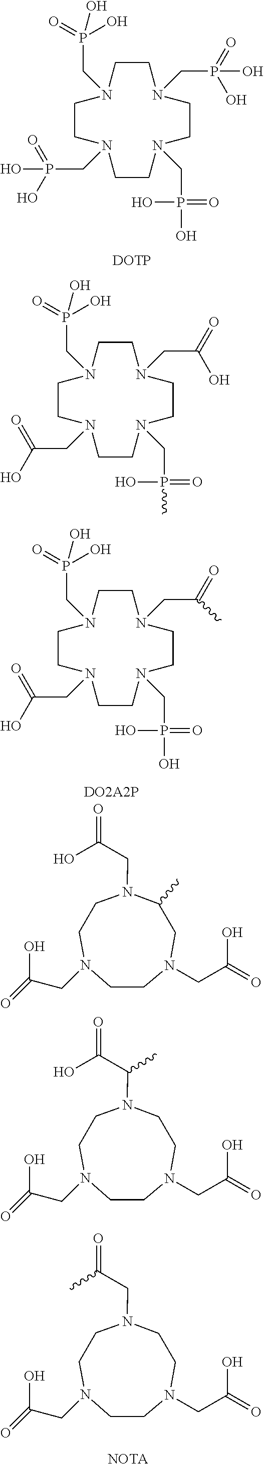

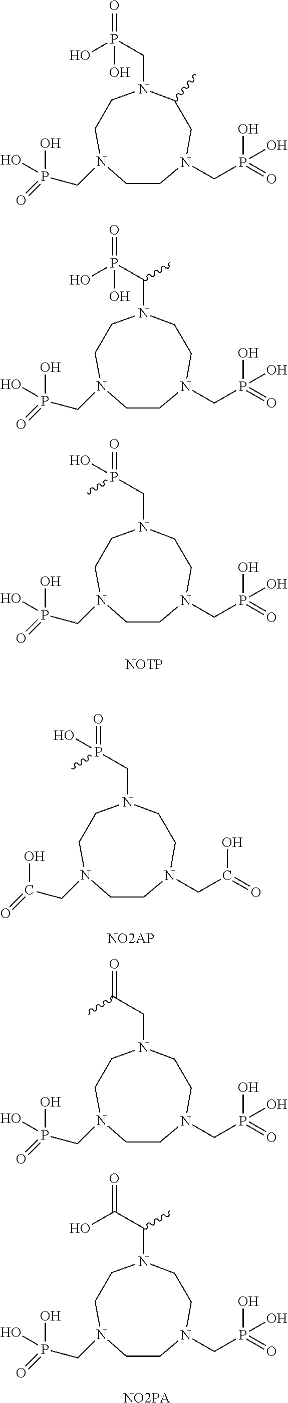

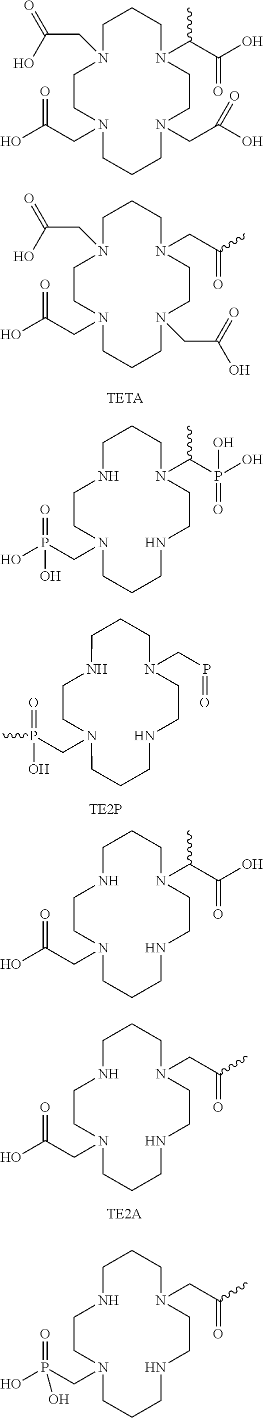

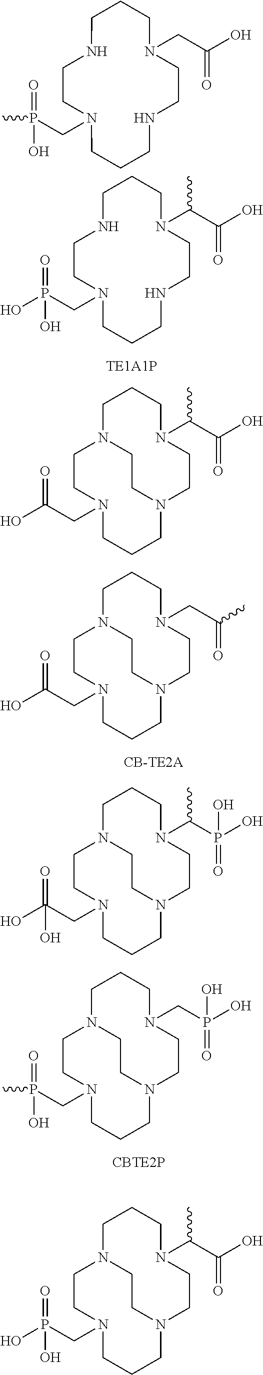

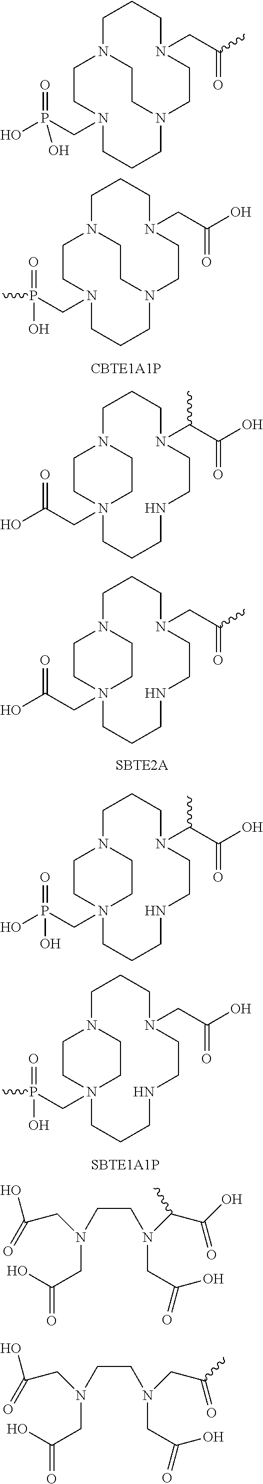

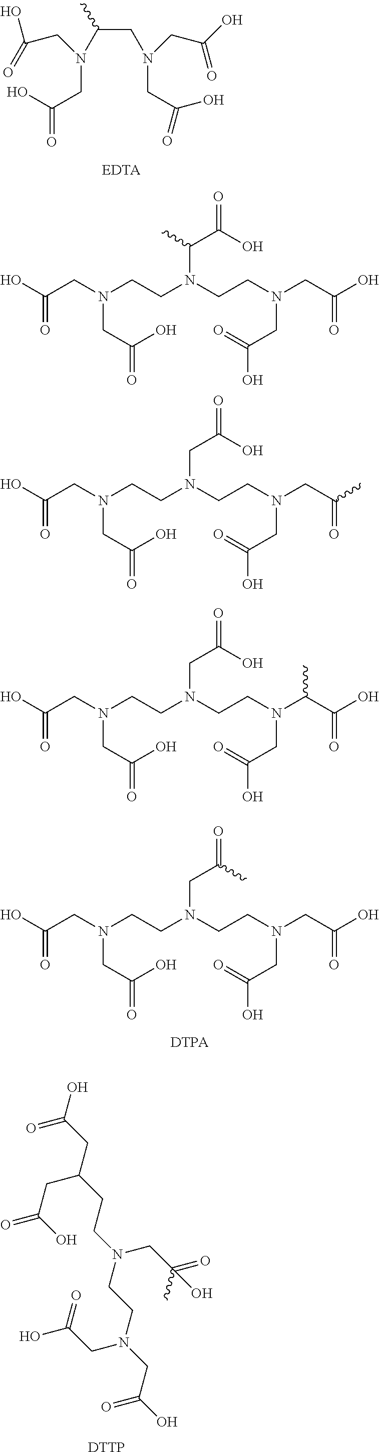

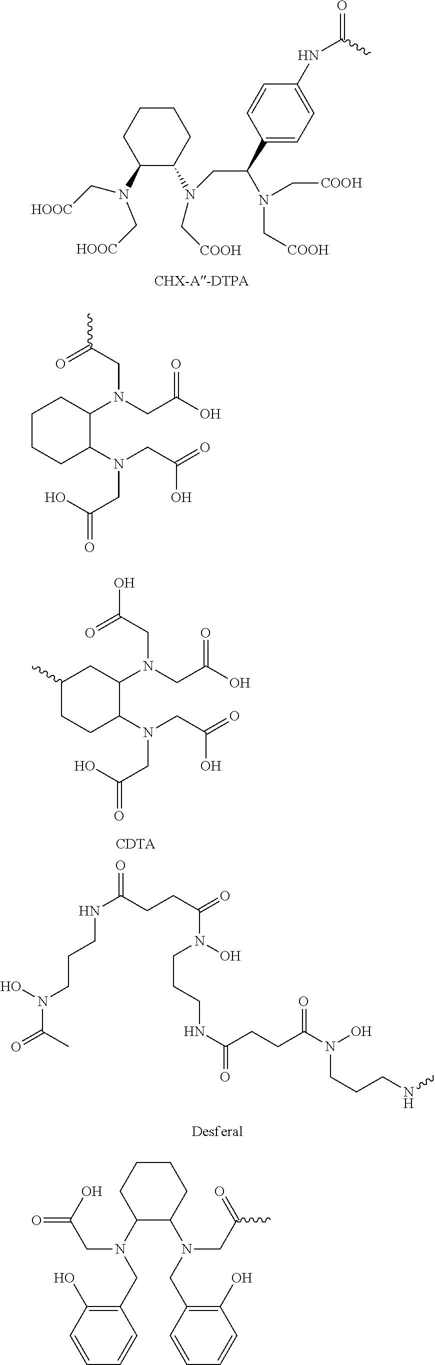

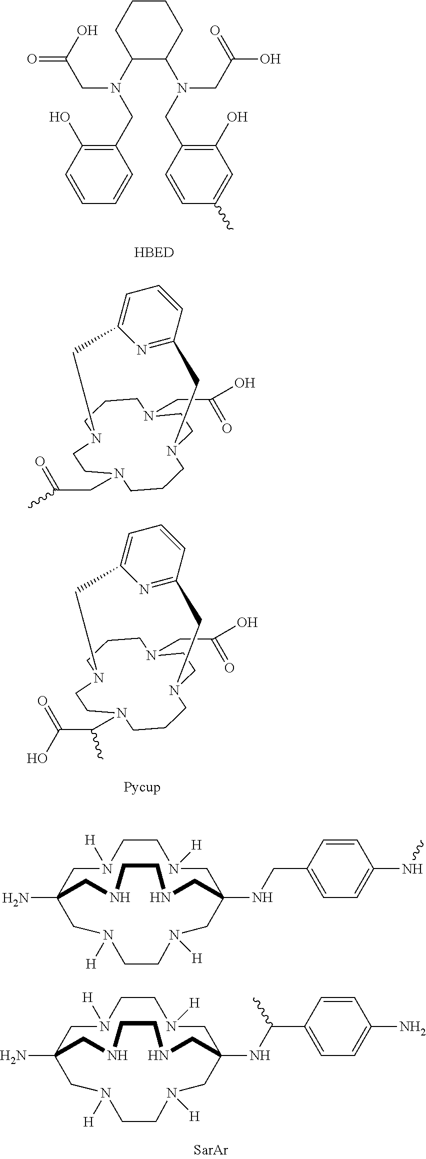

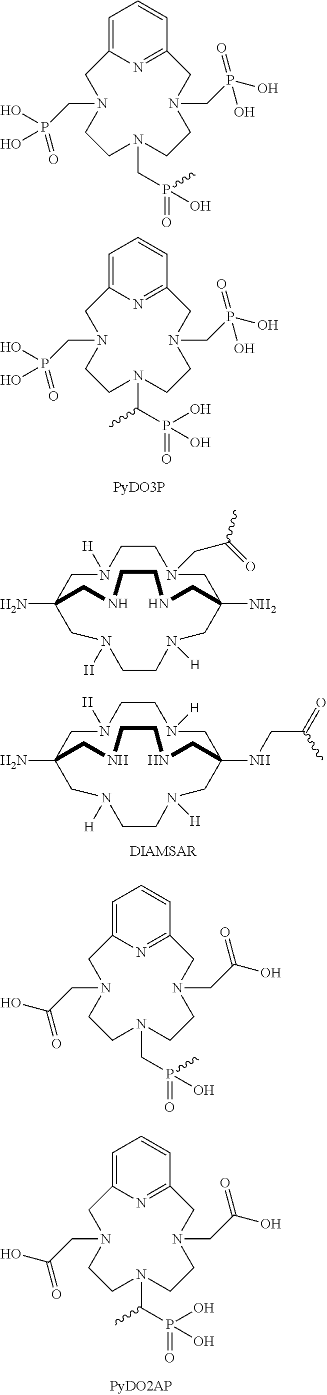

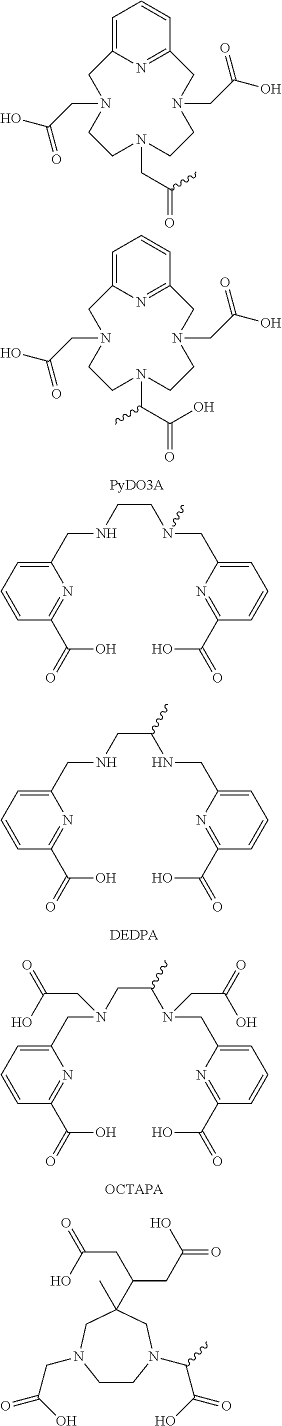

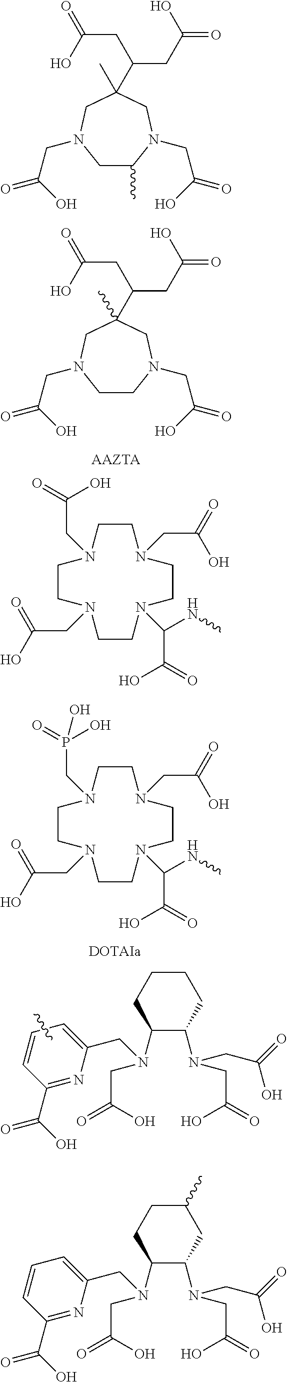

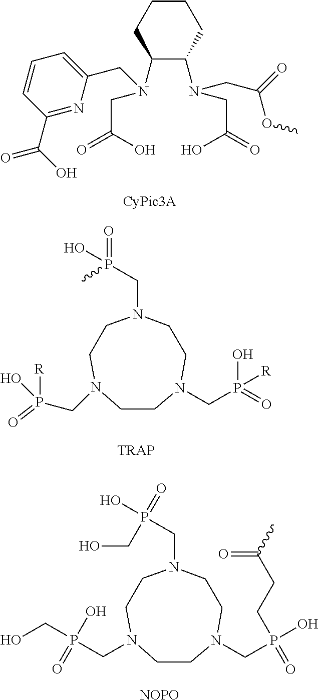

In such embodiments, the metal ion can be Gd³⁺, Mn³⁺, Mn²⁺, Fe³⁺, Ce³⁺, Pr³⁺, Nd³⁺, Eu³⁺, Eu²⁺, Tb³⁺, Dy³⁺, Er³⁺, Ho³⁺, Tm³⁺, Yb³⁺, Cr³⁺, or an ion of a radioisotope selected from the group consisting of ⁶⁷Ga, ⁶⁸Ga, Al-¹⁸F, ⁶⁴Cu, ¹¹¹In, ⁵²Mn, ⁸⁹Zr, ⁸⁶Y, ²⁰¹TI, ^{94m}Tc, and ^{99m}Tc; Y can be NH₂ or O; X can be CH₂ or O; L can be —CH₂CH₂—, —NHCH₂—, —CH₂—Ph—, or —CH₂CH₂CH₂—; and each of Rₐ and R_{b}, independently, can be H or CH₃. Examples of such compounds include:

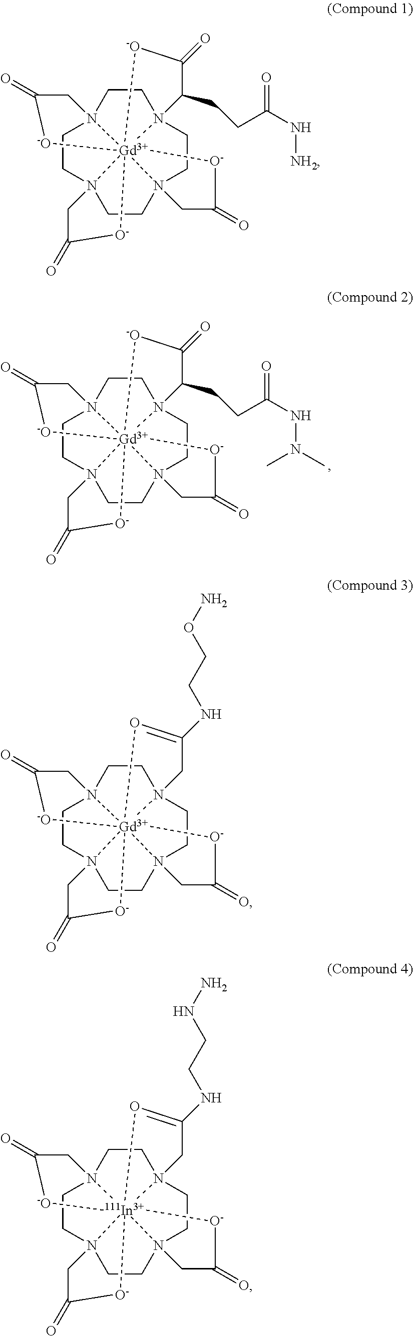

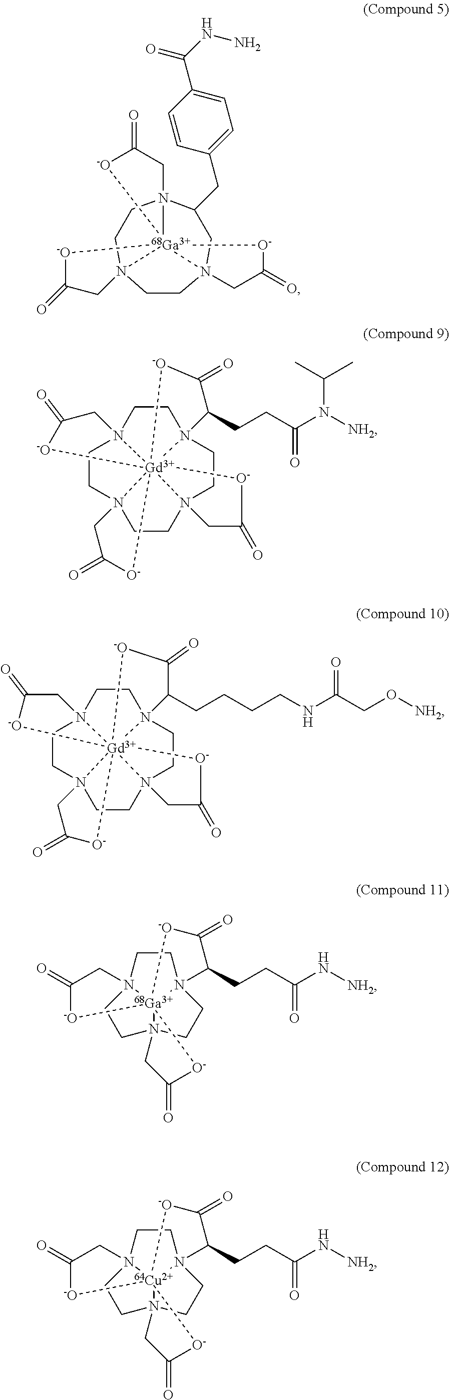

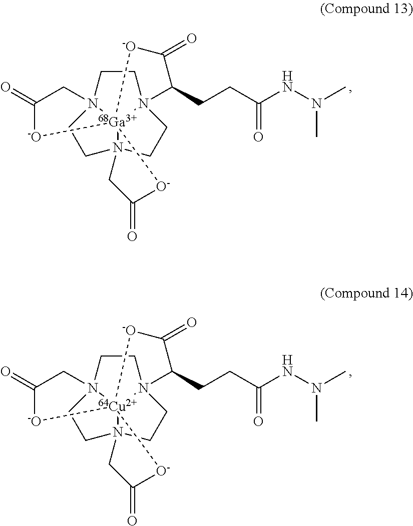

or a pharmaceutically acceptable salt thereof.

In some embodiments, the compound is selected from

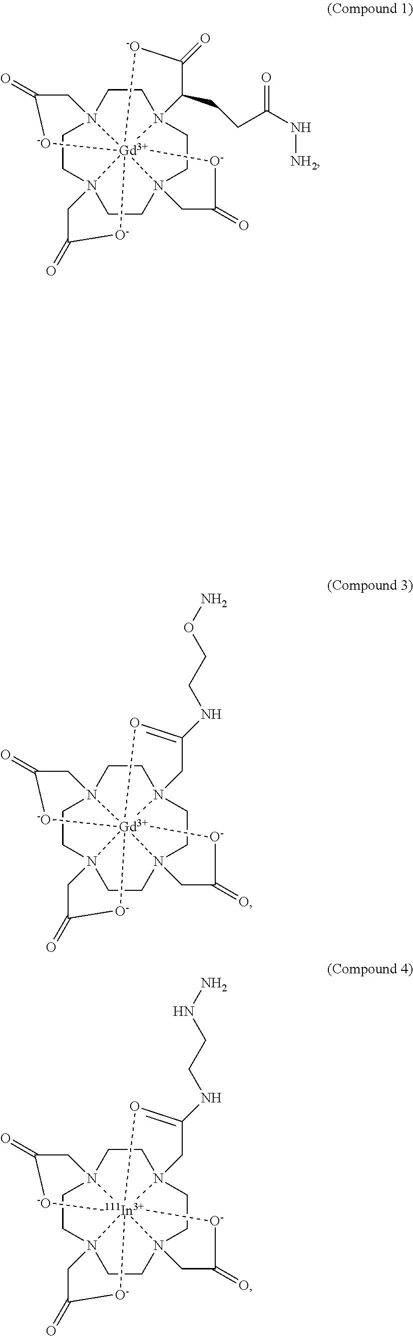

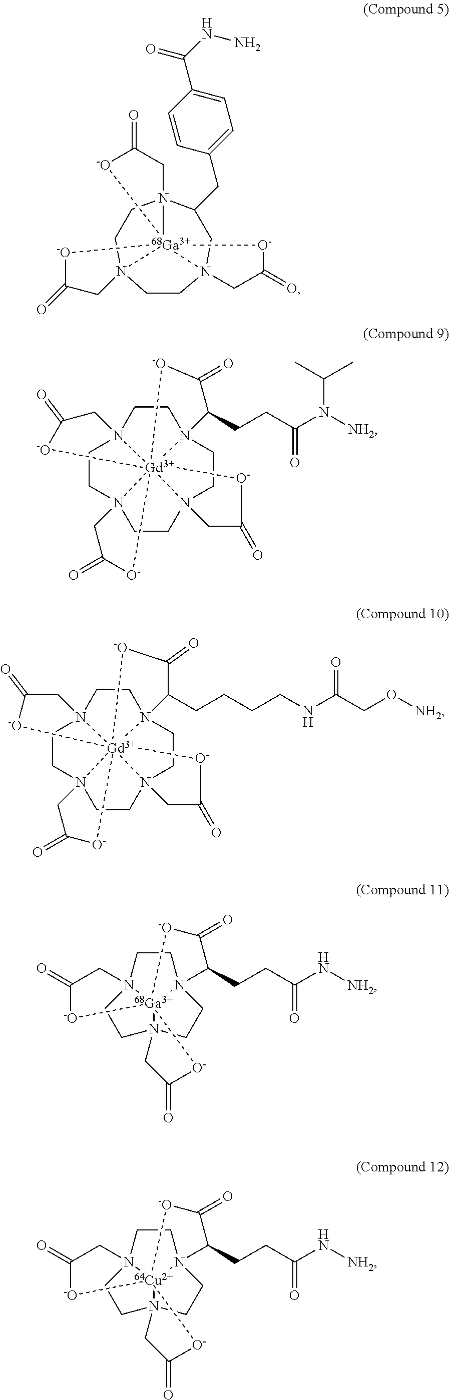

or a pharmaceutically acceptable salt thereof.

In some embodiments, Z further comprises a water molecule complexed with the metal ion. Examples of such compounds include:

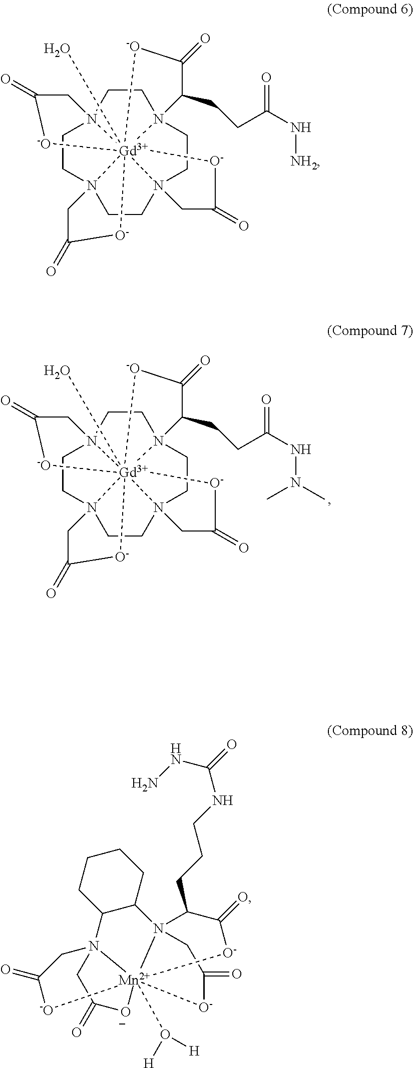

or a pharmaceutically acceptable salt thereof.

In some embodiments, the compound wherein Z further comprises a water molecule complexed with the metal ion is selected from:

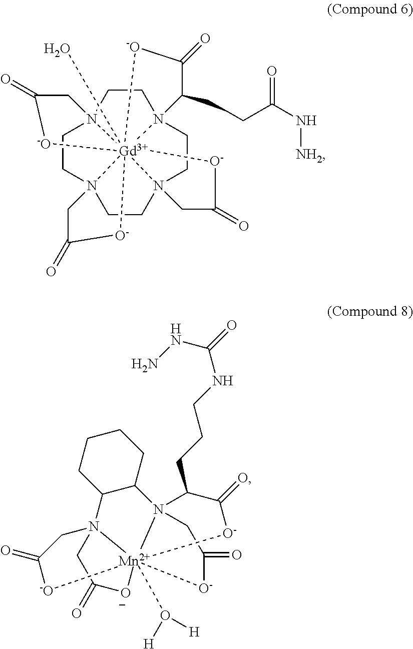

or a pharmaceutically acceptable salt thereof.

The compounds of formula (I) and/or (la) described herein above include the compounds themselves, as well as their salts, prodrugs, and solvates, if applicable. A salt, for example, can be formed between an anion and a positively charged group (e.g., amino) on a compound of formula (I). Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a compound of formula (I) and/or (Ia). Suitable cations include sodium ion, potassium ion, magnesium ion, calcium ion, and an ammonium cation such as tetramethylammonium ion or N-methylglucammonium ion.

The compound of formula (I) and/or (Ia) also include those salts containing quaternary nitrogen atoms. Examples of prodrugs include esters, amides, carbamates, carbonates, and other pharmaceutically acceptable derivatives, which, upon administration to a subject, are capable of providing a compound of formula (I) and/or (Ia). A solvate refers to a complex formed between a compound of formula (I) and/or (Ia) and a pharmaceutically acceptable solvent. Examples of pharmaceutically acceptable solvents include water, ethanol, isopropanol, ethyl acetate, acetic acid, and ethanolamine.

The compounds of formula (I) and/or (Ia) mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

The compounds of formula (I) and/or (Ia) described herein can be prepared by methods well known in the art. The Examples below provide detailed descriptions of how Compounds described above were prepared.

Other compounds of formula (I) and/or (Ia) can be prepared using other suitable starting materials through the synthetic routes described in the Examples or other synthetic routes known in the art. The methods described herein may also include additional steps, either before or after the steps described specifically herein, to add or remove suitable protecting groups in order to ultimately allow synthesis of a compound of formula (I) and/or (Ia). In addition, various synthetic steps may be performed in an alternate sequence or order to give the desired compounds. Synthetic chemistry transformations and protecting group methodologies (protection and deprotection) useful in synthesizing applicable compounds of formula (I) and/or (Ia) are known in the art and include, for example, those described in R. Larock, Comprehensive Organic Transformations, VCH Publishers (1989); T. W. Greene and P. G. . Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Also within the scope of this invention is a pharmaceutical composition containing an effective amount of at least one compound of formula (I) and/or (Ia) and a pharmaceutical acceptable carrier.

### Methods

Lysyl oxidase (LOX) and LOX-like enzymes are extracellular enzymes involved in cross linking collagen and/or elastin fibrils. These enzymes catalytically oxidize lysine amino groups to aldehydes and the aldehydes then undergo non-catalytic condensation reactions with other amino acid side chains (or another oxidized lysine) to produce stable covalent crosslinks. The compounds of the disclosure (e.g., a compound of formula (I) and/or (Ia)) target these aldehydes that are generated by LOX by using an imaging agent (Gd, Mn, nuclear, etc.) with a group such as a hydrazide (—NH—NH₂) or amino-oxy (—O—NH₂) that would undergo a condensation reaction with an aldehyde to form a neutral imine-containing product. Since aldehydes are rare in the body, and because the compounds of the disclosure do not readily penetrate cells, the compounds are selective for tissue with high levels of LOX activity in the extracellular matrix. LOX activity is upregulated in active fibrosis (fibrogenesis), in arterial remodeling, and in many cancers. Diseases having a strong fibroproliferative component and may comprise increased LOX activity include, but are not limited to, heart failure, heart attack, end stage renal disease, all forms of hepatitis, pulmonary fibrosis, scleroderma, atherosclerosis, and many aggressive cancers.

One aspect of the disclosure is a method for assessing LOX activity in an extracellular matrix of a biological sample, in a tissue, in a tumor, and/or in a mammal using an imaging agent. In some embodiments, an imaging agent comprises a hydrazide (—NR—NH₂) or amino-oxy (—O—NH₂) group, wherein R is H, C₁-C₁₀ alkyl, C₂-C₁₀ alkenyl, C₂-C₁₀ alkynyl, or aryl, can be used to assess LOX activity in an extracellular matrix of a biological sample, in a tissue, in a tumor, and/or in a mammal. In some embodiments, the imaging agent is a compound of formula (I) and/or (Ia), or a pharmaceutically acceptable salt thereof.

This disclosure provides for method of imaging an extracellular matrix of a biological sample, comprising contacting the extracellular matrix with an imaging agent as described herein. In some embodiments, the extracellular matrix comprises a plurality of cells. Without wishing to be bound by theory, the compounds of the disclosure (e.g., a compound of formula (I) and/or (Ia)) can bind and react selectively with an aldehyde generated by LOX in the extracellular matrix closely surrounding a cell. In some embodiments, an imaging agent comprises a hydrazide (—NR—NH₂) or amino-oxy (—O—NH₂) group, wherein R is H, C₁-C₁₀ alkyl, C₂-C₁₀ alkenyl, C₂-C₁₀alkynyl, or aryl, can be used to image a cell. In some embodiments, the imaging agent is a compound of formula (I) and/or (Ia), or a pharmaceutically acceptable salt thereof. In some embodiments, the contacting is in vitro. In some embodiments, the contacting is in vivo. In some embodiments, the cell is a blood cell, a cancer cell, an immune cell (e.g., a macrophage cell), an epithelial cell (e.g., a skin cell), a bacterial cell, or a virus-infected cell.

In some embodiments, the cell is a cancer cell. In some embodiments, the cancer cell is selected from a breast cancer cell, a colon cancer cell, a leukemia cell, a bone cancer cell, a lung cancer cell, a bladder cancer cell, a brain cancer cell, a bronchial cancer cell, a cervical cancer cell, a colorectal cancer cell, an endometrial cancer cell, an ependymoma cancer cell, a retinoblastoma cancer cell, a gallbladder cancer cell, a gastric cancer cell, a gastrointestinal cancer cell, a glioma cancer cell, a head and neck cancer cell, a heart cancer cell, a liver cancer cell, a pancreatic cancer cell, a melanoma cancer cell, a kidney cancer cell, a laryngeal cancer cell, a lip or oral cancer cell, a lymphoma cancer cell, a mesothioma cancer cell, a mouth cancer cell, a myeloma cancer cell, a nasopharyngeal cancer cell, a neuroblastoma cancer cell, an oropharyngeal cancer cell, an ovarian cancer cell, a thyroid cancer cell, a penile cancer cell, a pituitary cancer cell, a prostate cancer cell, a rectal cancer cell, a renal cancer cell, a salivary gland cancer cell, a sarcoma cancer cell, a skin cancer cell, a stomach cancer cell, a testicular cancer cell, a throat cancer cell, a uterine cancer cell, a vaginal cancer cell, and a vulvar cancer cell.

The present disclosure further provides a method for imaging a tissue, comprising administering to the tissue an imaging agent. In some embodiments, an imaging agent comprises a hydrazide (—NR—NH₂) or amino-oxy (—O—NH₂) group, wherein R is H, C₁-C₁₀ alkyl, C₂-C₁₀ alkenyl, C₂-C₁₀ alkynyl, or aryl, can be used to image a tissue. In some embodiments, the imaging agent is a compound of formula (I) and/or (Ia), or a pharmaceutically acceptable salt thereof. Tissues that can be imaged using the methods of the disclosure can be any of breast tissue, colon tissue, bone tissue, lung tissue, bladder tissue, brain tissue, bronchial tissue, cervical tissue, colorectal tissue, endometrial tissue, ependymal tissue, eye tissue, gallbladder tissue, gastric tissue, gastrointestinal tissue, neck tissue, heart tissue, liver tissue, pancreatic tissue, kidney tissue, laryngeal tissue, lip or oral tissue, nasopharyngeal tissue, oropharyngeal tissue, ovarian tissue, thyroid tissue, penile tissue, pituitary tissue, prostate tissue, rectal tissue, renal tissue, salivary gland tissue, skin tissue, stomach tissue, testicular tissue, throat tissue, uterine tissue, vaginal tissue, and vulvar tissue. In some embodiments, the tissue is a liver, lung, heart or kidney tissue.

Fibrotic diseases show an enhanced level of LOX expression and/or activity that has been observed by numerous investigators. For example, Barker, H. E. et al. Nature Reviews Cancer 2012, 12, page 543 in Table 1 details enhanced expression and/or activity of one or more LOX family members in atherosclerosis, scleroderma (breast, lung, and/or tongue), liver cirrhosis, Alzheimer's dementia, non-Alzheimer's dementia, Wilson's disease, primary biliary cirrhosis, glaucoma, pseudoexfoliation syndrome, endometriosis, lung fibrosis, liver fibrosis, and heart failure. Imaging agents as described herein are useful for the visualization of affected tissues in fibrotic diseases. In some embodiments, the fibrotic disease is selected from the group consisting of: pulmonary fibrosis, chronic obstructive pulmonary disease, pulmonary arterial hypertension, heart failure, hypertrophic cardiomyopathy, myocardial infarction, atrial fibrillation, diabetic nephropathy, systemic lupus erythematosus, polycystic kidney disease, glomerulonephritis, end stage renal disease, nonalcoholic steatohepatitis, alcoholic steatohepatitis, hepatitis C virus infection, hepatitis B virus infection, primary sclerosing cholangitis, inflammatory bowel disease, scleroderma, atherosclerosis, glaucoma, diabetic retinopathy, radiation induced fibrosis, surgical adhesions, cystic fibrosis, and cancer. For example, the fibrotic disease can be idiopathic pulmonary fibrosis.

Cancers may arise from any cell type. Such cancers include, but are not limited to, a breast cancer, a colon cancer, a leukemia, a bone cancer, a lung cancer, a bladder cancer, a brain cancer, a bronchial cancer, a cervical cancer, a colorectal cancer, an endometrial cancer, an ependymoma, a retinoblastoma, a gallbladder cancer, a gastric cancer, a gastrointestinal cancer, a glioma, a head and neck cancer, a heart cancer, a liver cancer, a pancreatic cancer, a melanoma, a kidney cancer, a laryngeal cancer, a lip or oral cancer, a lymphoma, a mesothioma, a mouth cancer, a myeloma, a nasopharyngeal cancer, a neuroblastoma, an oropharyngeal cancer, an ovarian cancer, a thyroid cancer, a penile cancer, a pituitary cancer, a prostate cancer, a rectal cancer, a renal cancer, a salivary gland cancer, a sarcoma, a skin cancer, a stomach cancer, a testicular cancer, a throat cancer, a uterine cancer, a vaginal cancer, and a vulvar cancer. In some embodiments, the compounds of the disclosure (e.g., a compound of formula (I) and/or (Ia)) is useful to image a cancer selected from a breast cancer, a colon cancer, a bone cancer, a lung cancer, a bladder cancer, a brain cancer, a bronchial cancer, a cervical cancer, a colorectal cancer, an endometrial cancer, an ependymoma, a retinoblastoma, a gallbladder cancer, a gastric cancer, a gastrointestinal cancer, a glioma, a head and neck cancer, a heart cancer, a liver cancer, a pancreatic cancer, a melanoma, a kidney cancer, a laryngeal cancer, a lip or oral cancer, a mesothioma, a mouth cancer, a myeloma, a nasopharyngeal cancer, a neuroblastoma, an oropharyngeal cancer, an ovarian cancer, a thyroid cancer, a penile cancer, a pituitary cancer, a prostate cancer, a rectal cancer, a renal cancer, a salivary gland cancer, a sarcoma, a skin cancer, a stomach cancer, a testicular cancer, a throat cancer, a uterine cancer, a vaginal cancer, and a vulvar cancer.

A number of reports have correlated increased LOX activity in cancers. See, for example, Cox, T. R. et al. Cancer Research 2013, 73(6), 1721-1732; Cox, T. R. and Erler, J. T. Carcinogenesis & Mutagenesis 2013, S13; Erler, J. T. et al. Nature 2006, 440, 1222-1226; Mayorca-Guiliani, A. and Erler, J. T. OncoTargets and Therapy 2013, 6, 1729-1735; Naba, A. et al. BMC Cancer 2014,14, 518-529; Barker, H. E. et al. Nature Reviews Cancer 2012,12, 540-552; Moon, H.-J. et al. Bioorganic Chemistry 2014, 57, 231-241, each of which is hereby incorporated by reference in its entirety. On page 235, Moon et al. states:

The correlation between LOXL2 expression and tumor progression is dependent upon tissue type. LOXL2 expression is decreased in ovarian tumors. However, increased LOXL2 expression is associated with poor prognosis in patients with colon and esophageal tumors, as well as oral squamous cell carcinomas, laryngeal squamous cell carcinomas, and head and neck squamous cell carcinomas. Additionally, increased LOXL2 expression has been found to promote gastric cancer and breast cancer metastasis. Some highly invasive human breast cancer cell lines are reported to have elevated levels of LOXL2 mRNA. (citations removed)

Members of the LOX family has been implicated in epithelial cell plasticity and tumor progression, including in small cell carcinoma (SCC). See, e.g., Cano, A. et al. Future Oncology 2012, 8(9), 1095-1108, hereby incorporated by reference in its entirety. Cano et al. states on page 1101:

[I]ncreased mRNA levels of ZOX have been observed in oral SCC, head and neck cancer, lung adenocarcinoma and breast cancer. In fact, LOX can be considered a poor prognostic factor in lung carcinoma. Polymorphic variants of LOX have also recently been found to be with increased risk of ovarian carcinoma. LOXL1 expression has also been detected in metastatic breast cancer cells and correlated with increased malignant potential. By contrast, epigenetic silencing of LOXL1 and LOXL4 genes has been observed in bladder carcinoma, leading to the proposal that they may act as tumor suppressors in this specific tumor type. Despite the limited information available regarding LOXL3 in human cancer samples, LOXL3 seems to be overexpressed in some specific tumor cell lines, (citations removed)

Other reports have correlated increased LOX expression in solid tumors and colorectal cancer (CRC). See, for example, Cox, T. R.; Erler, J. T. The American Journal of Physiology—Gastrointestinal and Liver Physiology 2013, 305, G659-G666, hereby incorporated by reference in its entirety. Cox and Erler at page G664:

The importance of LOX in solid tumors in general and in CRC is beyond doubt. Its implication in cell proliferation, invasion, and metastasis, driving angiogenesis and malignant transformation, has elevated it to a position as a viable target for therapeutic intervention. Indeed, cancer cells expressing high levels of LOX protein have an increased propensity to proliferate, invade, and metastasize in multiple solid tumor models, and there is compelling evidence from several laboratories to suggest that, in CRC, targeting LOX not only inhibits cancer cell invasion and metastasis but also reduces tumor angiogenesis since LOX regulates multiple signaling networks.

Thus increased LOX activity may be useful for the imaging and/or diagnosis in a number of diseases, such as cancers.

In general, the compounds of formula (I) and/or (Ia) described herein can be used in an imaging method for diagnosis of disorders, such as fibrosis (e.g., liver fibrosis, renal fibrosis, pulmonary fibrosis, uterine fibrosis, skin fibrosis, or cardiac fibrosis), fibrogenesis, atherosclerosis, myocardial infarct, or cancer (e.g., lung, breast, colorectal, primary liver, head and neck, or pancreatic cancer). The method includes administering to a mammal (e.g., a human) a compound of formula (I) and/or (Ia) (e.g., those in which each of R₁ and R₂ is H) and acquiring an image of a tissue (e.g., a liver, lung, heart, breast, uterine, prostate, skin, or kidney tissue) of the mammal after administration of the compound. The effective amount of the compound of formula (I) and/or (Ia) used in such a method will vary, as recognized by those skilled in the art, depending on the types of diseases to be diagnosed, route of administration, excipient usage, and the possibility of co-usage with other agents.

In some embodiments, the method can further include acquiring an image of the tissue of the mammal before administration of the compound. In such embodiments, the method can further include evaluating the differences between the images acquired before and after administration of the compound to determine whether the tissue is fibrotic.

Various imaging techniques can be used with the compounds of the disclosure and are known in the art. Imaging techniques include, but are not limited to, positron emission tomography (PET), single photon emission computed tomography (SPECT), computed tomography (CT), planar scintigraphy, and magnetic resonance imaging (MRI). Persons skilled in the art would recognize how to match the appropriate imaging agent with the appropriate imaging technique (e.g., those comprising ⁶⁴Cu, ⁶⁸Ga, ¹⁸F, ⁸⁶Y, and ^{94m}Tc are useful for PET imaging, those comprising ^{99m}Tc, ⁶⁷Ga, ¹¹¹In, and ²⁰¹TI are useful for SPECT imaging, those comprising Gd³⁺, Mn³⁺, Mn³⁺, Fe³⁺, Ce³⁺, Pr³⁺, Nd³⁺, Eu³⁺, Eu²⁺, Tb³⁺, Dy³⁺, Er³⁺, Ho³⁺, Tm³⁺, Yb³⁺, Cr³⁺ are useful for MRI).

In some embodiments, PET and SPECT imaging agents result in fibrotic tissue or tumor to have higher activity (signal intensity) than adjacent tissue. In some embodiments, the image taken with the target tissue or organ is compared to a reference value. For PET, a standardized uptake value (SUV) can be obtained, and a previously determined value would be indicative of fibrosis.

For MRI, the appropriate compounds of the disclosure (e.g., a compound of formula (I) and/or (Ia)) can change the MRI signal compared to the signal in an image taken before the probe is injected. Regions of fibrosis can have a greater change in signal intensity (signal intensity higher on T1-weighted image, lower on T2-weighted image). The contrast between fibrotic and adjacent tissue can be higher (difference between signal in fibrotic tissue and signal in adjacent signal). Alternately, the change in relaxation time T1 or T2 can be measured after injection of the probe. Changes in relaxation rate (1/T1 or 1/T2) greater than a certain value would indicate fibrosis. In some embodiments, the method can include (a) acquiring a T1-weighted image of a tissue of the mammal at from about 1 minute to about 10 minutes after administration of the compound of formula (I) and/or (Ia). In such embodiments, the method can further include (b) acquiring a second T1-weighted image of the tissue of the mammal at a time from about 10 minutes to about 2 hours after administration of the compound of formula (I) and/or (Ia); and evaluating differences between the images acquired in steps (a) and (b), where a non-fibrotic pathology exhibits greater loss in enhancement from the image collected in step (a) to that in step (b) as compared to a fibrotic pathology.

Without wishing to be bound by theory, it is believed that lysyl oxidase (LOX) and lysyl oxidase-like enzymes (LOXL-n) oxidize peptidyl lysine in collagen and elastin substrates to residues of α-aminoadipic-δ-semialdehyde. The peptidyl aldehydes can then undergo spontaneous condensations with unreacted ε-amino groups and with neighboring aldehyde functions, thus forming covalent cross-linking which converts elastin and collagen into insoluble fibers. Without wishing to be bound by theory, it is believed that the compounds of formula (I) and/or (Ia) (e.g., those in which each of R₁ and R₂ is H) can react with the peptidyl aldehydes generated by the action of LOX on collagen to attach the compound to such a collagen. Without wishing to be bound by theory, it is believed that the imaging group in the compounds of formula (I) and/or (Ia) (i.e., the cyclic structure that forms a metal complex) can then be used to generate MR images that have enhanced MR signals.

In some embodiments, the compounds of formula (I) and/or (Ia) may be used in the same manner as a conventional MRI diagnostic composition and are useful for imaging extracellular matrix components of an organ. For example, a compound of formula (I) and/or (Ia) is administered to a patient (e.g., a mammal such as a human) and an MR image of the patient is acquired. Generally, the clinician will acquire an image of an area having the extracellular matrix component that is targeted by the agent. For example, the clinician may acquire an image of the heart, lung, liver, kidney, or another organ or tissue type where the compound of formula (I) and/or (Ia) targets collagen or locations of abnormal collagen or elastin accumulation in a disease state. The clinician may acquire one or more images at a time before, during, or after administration of the compound of formula (I) and/or (Ia). Other techniques of using a MRI diagnostic composition have been described, e.g., U.S. Application Publication Nos. 2008/0044360 and 2013/0309170.

To practice the method disclosed herein, a composition having one or more compounds of formula (I) and/or (Ia) described above can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more compounds of formula (I) and/or (Ia) described above can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of a compound of the invention. Examples of other carriers include colloidal silicon oxide, magnesium stearate, cellulose, sodium lauryl sulfate, and D&C Yellow # 10.

The compounds of formula (I) and/or (Ia) described above can be preliminarily screened for their efficacy in diagnosis of a disorder by in vivo assays (see Example 3 below). Other methods will also be apparent to those of ordinary skill in the art.

The contents of all publications cited herein (e.g., patents, patent application publications, and articles) are hereby incorporated by reference in their entirety.

### EXAMPLES

The following examples are illustrative and not intended to be limiting.

### Example 1

### Preparation of Compound 1:2-(R)-2-(4,7,10-tris-carboxymethyl-1,4,7,10-tetraazacyclododec-1-yl)-pentanedioic acid-1-hydrazide gadolinium Complex

### General Procedures

### Probe Synthesis

2-(R)-2-(4,7,10-tris tert-butylcarboxymethyl-1,4,7,10-tetraazacyclododec-1 -yl)-pentanedioic acid-1-tert-butylester (tBuDOTAGA) was obtained as described previously (Levy et al., Organic Process Research and Development, 2009, 13(3), 535). All other reactants and reagents were of commercial grade and were used without further purification.

### NMR

NMR spectra were recorded on a Yarian 500 NMR system equipped with a 5 mm broadband probe (¹H NMR: 499.81 MHz, ¹³C: 125.68 MHz, ³¹P: 207.33 MHz).

### Preparative HPLC

Purifications were performed using the following methods. Fractions containing product with a purity >95% were combined:

Method 1: Column: MetaChem Rechnologies Inc., Polaris C₁₈-A 10 μm 250×212 mm, flow rate: 25 ml/min, solvent A: 0.1% TFA in water, B: 0.1% TFA in MeCN, 5% B for 5 min, gradient to 30% B within 1 min followed by gradient to 55% in 10 min, gradient to 100% B within 1 min, plateau for 2 min and reequilibration for 6 min.

Method 2: Column: Restek, UltraAqueous C_{18, 5} μm 250×10 mm, flow rate: 5 ml/min, solvent A: NH₄OAc(10 mM, pH 6.9) in water, B: 0.1% TFA in MeCN/NH₄OAc(10 mM, pH 6.9) 9:1, 2% B for 4 min, gradient to 72% B within 11 min followed by gradient to 95% B in 1 min, plateau for 2 min and reequilibration for 2 min.

### HPLC-MS

HPLC-MS purity analysis was carried out on an Agilent 1100 system using the following methods:

Method A: column: Phenomenex Luna, C18(2), 100×2 mm, flow rate: 0.8 ml/min, UV detection at 220, 254 and 280 nm, 5% of MeCN (0.1% formic acid) in 0.1% formic acid for 1 min., then gradient to 95% MeCN (0.1% formic acid) in 9 min, 2 min. plateau, reequilibration for 2 min.

Method B: column: Restek, UltraAqueous C18, 5 μm 250×4.6 mm, flow rate: 0.8 ml/min, UV detection at 220, 254 and 280 nm, 5% of MeCN/NH₄OAc(10 mM, pH 6.9) 9:1 in ammonium formate (10 mm, pH 6.9) for 1 min., then gradient to 95% MeCN/NH₄OAc(10mM, pH 6.9) 9:1 in 9 min, 2 min. plateau, reequilibration for 2 min.

### UV Titration

Into a 1.5 mL quartz cuvette is placed 10 μL of ligand solution and 1 mL of an arsenazo III solution (10 μM arsenazo III in 0.15 M NH₄OAC buffer pH 7). The cuvette is placed into a UV/Vis spectrophotometer and zeroed at 656 nm. Aliquots of 10 μL of a 4.85 mM Pb(NO₃)₂ solution (or 0.485 mM solution close to the end point), are titrated into the cuvette until a positive absorbance is observed. A positive absorbance represents the end point of the titration.

### Method of Preparation of Compound 1

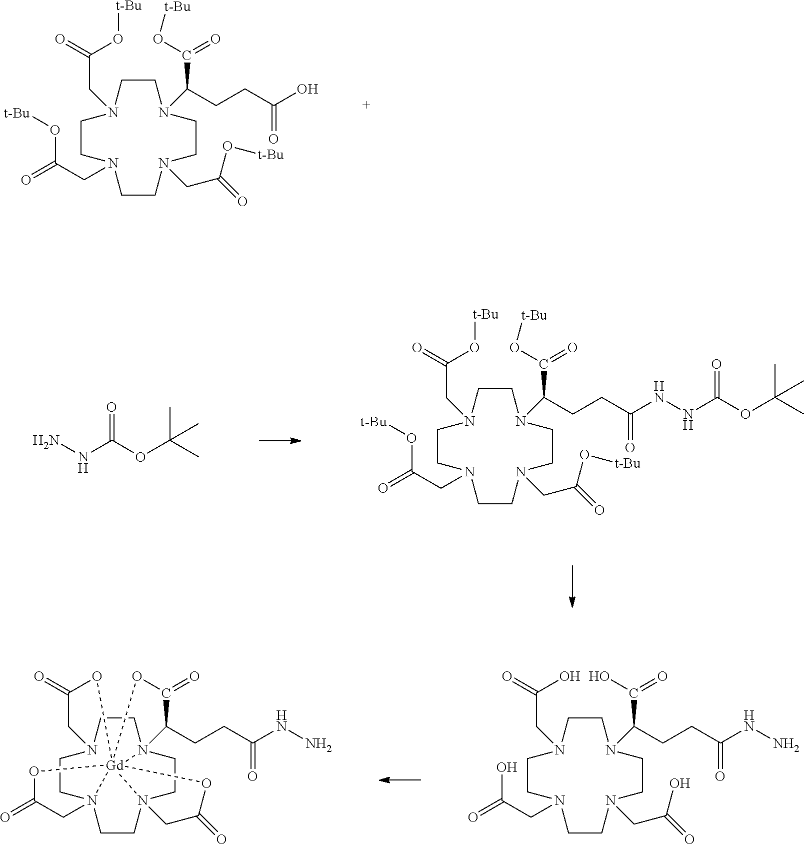

2-(R)-2-(4,7,10-tris tert-butlcarboxymethyl-1,4,7,10-tetraazacyclododec-1-yl)-pentanedioic acid-1-N′-tert-butoxycarbonyl-N-hydrazide

2-(R)-2-(4,7,10-tris tert-butylcarboxymethyl-1,4,7,10-tetraazacyclododec-1-yl)-pentanedioic acid-1-tert-butylester (500 mg, 713 μmol) and O-(7-azabenzotriazol-1-yl)-N,N,N′,N′-tetramethyluronium hexafluorophosphate (HATU, 325 mg, 856 μmol) were dissolved in dry DMF (25 ml). After 5 minutes, solid tert-butyl-semicarbazide (113 mg, 856 μmol) was added and stirring was continued for 24 hours. After the solvent was evaporated, the residue was purified using Method 1 to yield 574 mg (704 μmol, 98.7%) of a white solid product.

¹NMR (DMSO-d₆, 90° C.): 9.27 (br s, 1H), 8.21 (br s, 1H), 3.72-3.81 (m, 4H), 3.47-3.56 (m, 3H), 3.07-3.14 (m, 8H), 2.90-2.93 (m, 8H), 2.21 (m, 2H), 1.87-1.95 (m, 2H), 1.46, 1.44, 1.40 (3s, 45H). LC: Method A, t_{R}=2.55 minute. LC/MS (ESI+): C₄₀H₇₄N₆O₁₁: m/z (%): calcd 815.54 [MH⁺]; found: 815.45 (MH⁺).

2-(R)-2-(4,7,10-tris-carboxymethyl-1,4,7,10-tetraazacyclododec-1-yl)-pentanedioic acid-1-hydrazide

2-(R)-2-(4,7,10-tris tert-butylcarboxymethyl-1,4,7,10-tetraazacyclododec-1-yl)-pentanedioic acid-1-N′-tert-butoxycarbonyl-N-hydrazide (100 mg, 123 μmol) was dissolved in a mixture of TFA (1.5 ml), triisopropyl silane (90 μl) and 1-dodecathiol (90 μl). The mixture was stirred at room temperature overnight. The volatiles were removed in vacuum and the residue was re-dissolved in half-concentrated HCl. After the solution was stirred for 3 hours at room temperature, the solution was lyophilized.

The residue was re-dissolved in water and the pH was adjusted to 7 using ammonium hydroxide. After lypholization, the solid was re-dissolved in water (2.00 ml) and subjected to a UY titration using arsenazo III (see above) to determine the concentration of ligand (36 mM, 72 μmol, 58.5%).

¹ H NMR (D₂O, 80° C., pH9): 4.35 (d, J=16.7 Hz, 2H), 4.24 (d, J=16.7 Hz, 2H), 3.78-4.14 (m, 17H), 3.65 (br s, 2H), 2.95 (br s, 2H), 2.61 (br s, 1H), 2.47 (br s, 1H).

LC: Method B, t_{R}=2.51 minute. LC/MS (ESI+): C₄₀H₇₄N₆O₁₁: m/z (%): calcd 491.51 [MH⁺]; found: 491.35 (MH⁺).
2-(R)-2-(4,7,10-tris-carboxymethyl-1,4,7,10-tetraazacyclododec-1-yl)-pentanedioic acid-1-hydrazide gadolinium complex (Compound 1)

The stock solution of 2-(R)-2-(4,7,10-tris-carboxymethyl-1,4,7,10-tetraazacyclododec-1-yl)-pentanedioic acid-1-hydrazide obtained above was treated with GdCl₃*6H₂O (27.0 mg, 72.6 μmol) and the pH of the solution was adjusted to 6.2. After the solution was stirred for 1 hour, the ligand of MS-325 (55 mg, 73.0 μmol) was added and the pH of the mixture was maintained between 4 and 8. After 1 hour, the pH was adjusted to 7 and the solution lyophilized. The residue was dissolved in the aqueous eluent used in Method 2 and purified using this method to yield 30.0 mg (46.5 μmol, 64.1%) of the title compound after lyophilization. Both arsenazo III and xylenol orange test were negative demonstrating the absence of non-chelated Gd(III).

LC: Method B, t_{R}=3.73 minute. LC/MS (ESI+): C₁₉H₃₀GdN₆O₉: m/z (%): calcd 646.13 [MH⁺]; found: 646.20 (MH⁺).

### Example 2

### Preparation of Compound 2: (R)-2,2′,2″-(10-(1-carboxy-4-(2,2-dimethylhydrazinyl)-4-oxobutyl)-1,4,7,10-tetraazacyclododecane-1,4,7-triyl)triacetic acid Gadolinium complex

### General Procedure

### Preparative HPLC

Purifications were performed using the following methods. Fractions containing product with a purity >95% were combined:

Method 1: Column: Phenomenex Luna, C18(2) 10 μm 250×21.2 mm, flow rate: 18 ml/min, solvent A: 0.1% TFA in water, B: 0.1% TFA in MeCN, 5%B for 5 min, gradient to 30% B within 1 min followed by gradient to 75% in 10 min, gradient to 100% B within 1 min, plateau for 2 min and reequilibration for 6 min.

Method 2: Column: Restek, UltraAqueous C_{18, 5} μm 250×10 mm, flow rate: 4 ml/min, solvent A: 0.1% TFA in water, B: 0.1% TFA in MeCN, 2% B for 4 min, gradient to 72% B within 11 min followed by gradient to 95% B in 1 min, plateau for 2 min and reequilibration for 2 min.

### HPLC-MS

HPLC-MS purity analysis was carried out on an Agilent 1100 system using the following methods:

Method A: column: Phenomenex Luna, C18(2), 100×2 mm, flow rate: 0.8 ml/min, UV detection at 220, 254 and 280 nm, 5% of MeCN (0.1% formic acid) in 0.1% formic acid for 1 min., then gradient to 95% MeCN (0.1% formic acid) in 9 min, 2 min. plateau, reequilibration for 2 min.

Method B: column: Restek, UltraAqueous C18, 5 μm 250×4.6 mm, flow rate: 0.8 ml/min, UV detection at 220, 254 and 280 nm, 5% of MeCN/NH₄OAc(10 mM, pH 6.9) 9:1 in ammonium formate (10 mm, pH 6.9) for 1 min., then gradient to 95% MeCN/NH₄OAc(10 mM, pH 6.9) 9:1 in 9 min, 2 min. plateau, reequilibration for 2 min.

### Method of Preparation of Compound 2

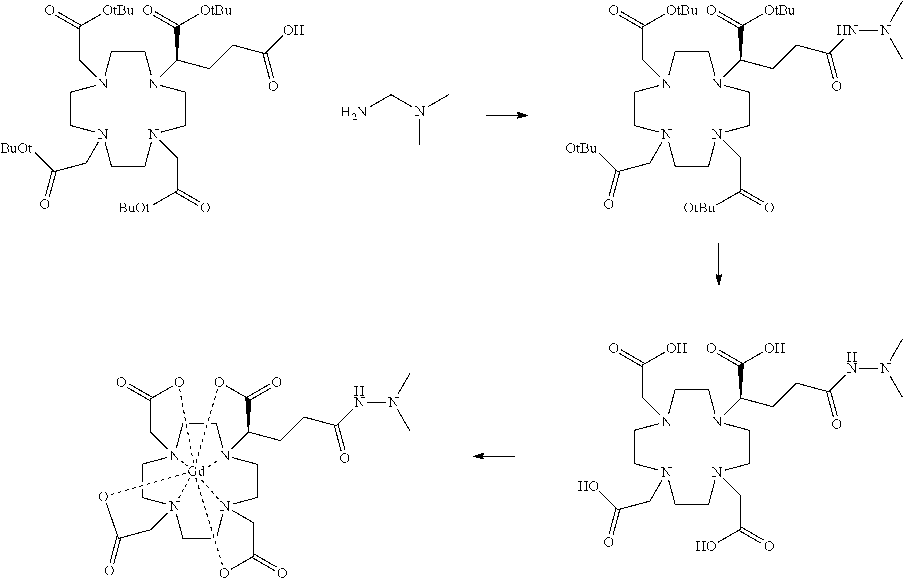

(R)-tri-tert-butyl 2,2′,2″-(10-(1-(tert-butoxy)-5-(2,2-dimethylhydrazinyl)-1,5-dioxopentan-2-yl)-1,4,7,10-tetraazacyclododecane-1,4,7-triyl)triacetate

(R)-5-(tert-butoxy)-5-oxo-4-(4,7,10-tris(2-(tert-butoxy)-2-oxoethyl)-1,4,7,10-tetraazacyclododecan-1-yl)pentanoic acid (500 mg, 713 μmol) and N,N′-Diisopropylcarbodiimide (DIC) (116 mg, 927 μmol) were dissolved in dichloromethane (25 mL). After 5 minutes, N,N-dimethylmethanediamine (70.5 μl, 927 μmol) was added and stirring continued for 24 hours. After the solvent was evaporated, the residue was purified using Method 1 to yield 350 mg (471 μmol, 66%) of a white solid product.

LC/MS: Method A, t_{R}=5.05 minute. LC/MS (ESI+): C₃₇H₇₀N₆O₉: m/z (%): calcd 743.99 [MH⁺]; found: 743.5 (MH⁺).
(R)-2,2′,2″-(10-(1-carboxy-4-(2,2-dimethylhydrazinyl)-4-oxobutyl)-1,4,7,10-tetraazacyclododecane-1,4,7-triyl)triacetic acid

(R)-5-(tert-butoxy)-5-oxo-4-(4,7,10-tris(2-(tert-butoxy)-2-oxoethyl)-1,4,7,10-tetraazacyclododecan-1-yl)pentanoic acid (350 mg, 471 μmol) was dissolved in a mixture of TFA (9 ml), triisopropyl silane (200 μl), 1-dodecanethiol (200 μl), water (200 μl) and methanesulfonic acid (200 μl). The mixture was stirred at room temperature for 2 hours. LC/MS showed complete reaction. The volatiles were removed in vacuum and the residue was re-dissolved in 5 ml of 1.0M HCl. After the solution was stirred for 3 hours at room temperature, the solution was lyophilized leaving 177.2 mg of white solid.

LC/MS: Method A, t_{R}=0.6 minute. LC/MS (ESI+): C₂₁H₃₈N₆O₉: m/z (%): calcd 519.56 [MH⁺]; found: 519.2 (MH⁺).
(R)-2,2′,2″-(10-(1-carboxy-4-(2,2-dimethylhydrazinyl)-4-oxobutyl)-1,4,7,10-tetraazacyclododecane-1,4,7-triyl)triacetic acid Gadolinium complex (Compound 2)

(R)-2,2′,2″-(10-(1-carboxy-4-(2,2-dimethylhydrazinyl)-4-oxobutyl)-1,4,7,10-tetraazacyclododecane-1,4,7-triyl)triacetic acid (50 mg, 96 μmol) was dissolved in water (10 mL) and the pH of the solution was adjusted to 7 with 0.1 N NaOH. The solution was treated with GdCl₃*6H₂O (35.0 mg, 92.2 μmol) and the pH was adjusted to 6. After the solution was stirred for 1 hour, EDTA (2 ml of 10 mM) was added and the pH was maintained between 4 and 8. After 1 hour, the pH was adjusted to 7 and the solution was loaded onto HPLC for purification using Method 2 to give 14 mg (20.8 μmol. 21.6%) of the title compound after lyophilization.

LC: Method B, t_{R}=4.5 minute. LC/MS (ESI+): C₂₁H₃₄GdN₆O₉: m/z (%): calcd 674.78 [MH⁺]; found: 675.0(MH⁺).

### Example 3

### Preparation of Compound 9 (gadolinium 2,2′, 2″-(10-(4-(2-((benzyloxy)carbonyl)-1-isopropylhydrazinyl)-1 -carboxy-4-oxobutyl)-1,4,7,10-tetraazacyclododecane-1,4,7-triyl)triacetate)

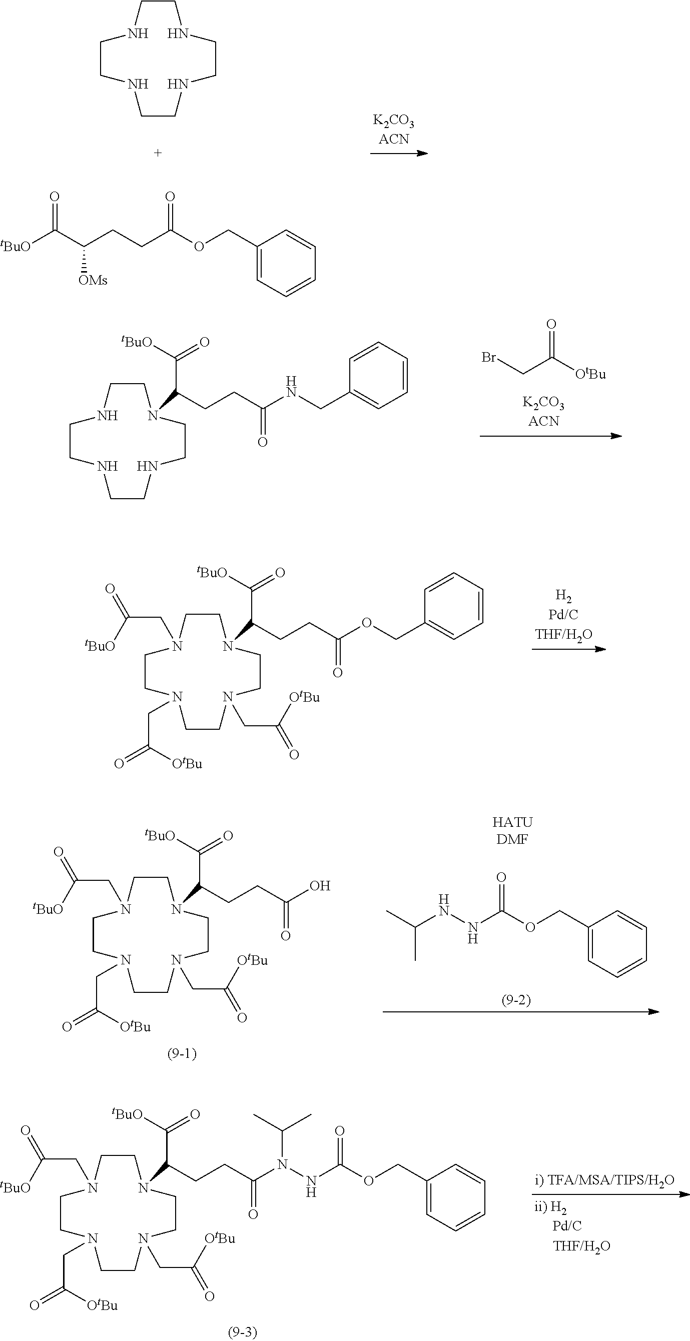

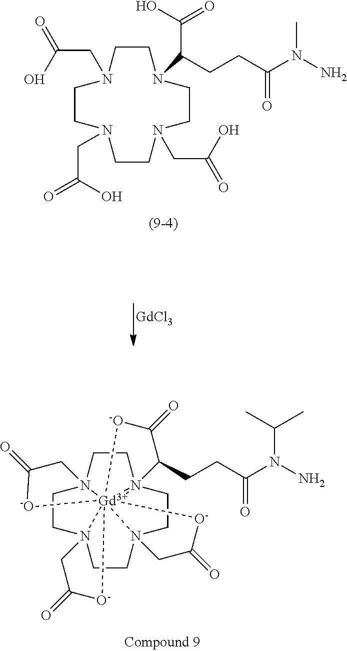

2-(R)-2-(4,7,10-tris tert-butylcarboxymethyl-1,4,7,10-tetraazacyclododec-1-yl)-pentanedioic acid-1-tert-butylester (9-1) and benzyl 2-isopropylhydrazine-1-carboxylate (9-2) were prepared according to literature protocols (Org. Process Res. Dev., 2009, 13, 535-542; ChemMedChem., 2013, 8, 1314-1321).

tri-tert-butyl 2,2′,2″-(10-(5-(2-((benzyloxy)carbonyl)-1-isopropylhydrazinyl)-1-(tert-butoxy)-1,5-dioxopcntan-2-yl)-1,4,7,10-tetraazacyclododecane-1,4,7-triyl)triacetate (9-3)

2-(R)-2-(4,7,10-tris tert-butylcarboxymethyl-1,4,7,10-tetraazacyclododec-1-yl)-pentanedioic acid-1-tert-butylester (9-1) (0.272 g, 0.38 mmol) and O-(7-azabenzotriazol-1-yl)-N,N,N′,N′-tetramethyluronium hexafluorophosphate (HATU, 0.162 g, 0.43 mmol) were dissolved in dry DMF (10 mL). After 5 min benzyl 2-isopropylhydrazine-1-carboxylate (9-2) (0.161 g, 0.77 mmol) was added and stirring continued for 24 h. The solvent was evaporated and the residue purified by preparative HPLC to yield 102 mg (0.115 mmol, 30%) of white solid product

¹H (d₆-DMSO): δ=7.37 (m, 5H), 5.13 (s, 2H), 4.52 (br. S, 1H), 3.80 (m, 4H), 3.45 (m, 3H), 3.12-2.92 (m, 16H), 2.28 (m, 2H), 1.92 (m, 2H), 1.51 (m, 36H), 1.04 (d, 6H) LC/MS (ESI+): C₄₆H₇₈N₆O₁₁: m/z (%) calcd 891.58 [MH⁺]; found 891.5 (MH⁺).
2, 2′, 2″-(10-(4-(2-((benzyloxy)carbonyl)-1-isopropylhydrazinyl)-1-carboxy-4-oxobutyl)-1,4,7,10-tetraazacyclododecane-1,4,7-triyl)triacetic acid (9-4)

Tri-tert-butyl 2,2′,2″-(10-(5-(2-((benzyloxy)carbonyl)-1-isopropylhydrazinyl)-1-(tert-butoxy)-1,5-dioxopentan-2-yl)-1,4,7,10-tetraazacyclododecane-1,4,7-triyl)triacetate (9-3) (40 mg, 44.9 μmol) was dissolved in a mixture of TFA (5 mL), triisopropyl silane (900 μL) and water (900 μL) and the mixture was stirred at room temperature overnight. The volatiles were removed in vacuo and the residue redissolved in water and the pH adjusted to 7 using ammonium hydroxide. After lyophilisation the residue was added to a slurry of palladium on carbon (dry, 1.9 mg, 10% by mass) in anhydrous methanol (5 mL). The mixture was subject to two cycles of vacuum and hydrogen purge and then stirred under an atmosphere of hydrogen for 12 h. After evacuating the system of hydrogen, celite was added and the slurry filtered through a MeOH-wet bed of celite. The filtrate was concentrated in vacuo and the solid was redissolved in water and subjected to a UV titration using arsenazo III to determine the concentration of ligand (7.67 mg, 14.4 μmol, 8.6 mM).

Step i) LC/MS (ESI+): C₃₀H₄₆N₆O₁₁: m/z calcd 667.33 [MH⁺]; found 667.4 (MH⁺)
Step ii) LC/MS (ESI+): C₂₂H₄₀N₆O₉: m/z calcd 533.29 [MH⁺]; found 533.3 (MH⁺)
2,2′,2″-(10-(4-(2-((benzyloxy)carbonyl)-1-isopropylhydrazinyl)-1-carboxy-4-oxobutyl)-1,4,7,10-tetraazacyclododecane-1,4,7-triyl)triacetic acid gadolinium complex (Compound 9)
The stock solution of 2,2′,2″-(10-(4-(2-((benzyloxy)carbonyl)-1-isopropylhydrazinyl)-1-carboxy-4-oxobutyl)-1,4,7,10-tetraazacyclododecane-1,4,7-triyl)triacetic acid (7.67 mg, 14.4 μmol) was treated with GdCl₃.6H₂O (5.45 mg, 14.66 μmol) and the pH adjusted to 6.8. After 12 h of stirring Na₂H₂EDTA (0.27 mg, 0.72 μmol) was added and the solution stirred for a further 2 h. The pH was adjusted to 7 and the solution purified by preparative HPLC to yield product (4.7 mg, 6.85 μmol, 48%). Both Arsenazo III and xylenol orange test were negative demonstrating the absence of non-chelated Gd(III).
LC/MS (ESI+): C₂₂H₃₆GdN₆O₉: m/z calcd 687.19 [MH⁺]; found 687.1 (MH⁺)

### Example 4

### Preparation of Compound 10 (gadolinium 2,2′,2″-(10-(5-(2-(aminooxy)acetamido)-1-carboxypentyl)-1,4,7,10-tetraazacyclododecane-1,4,7-triyl)triacetate)

Compounds tert-butyl 6-(((benzyloxy)carbonyl)amino)-2-bromohexanoate (10-1) and 2,5-dioxopyrrolidin-1-yl 2-(((tert-butoxycarbonyl)amino)oxy)acetate (10-6) were prepared according to literature protocols (PCT Int. Appl., 2006002873, 2006; J. Org. Chem., 2008, 73, 983-991).

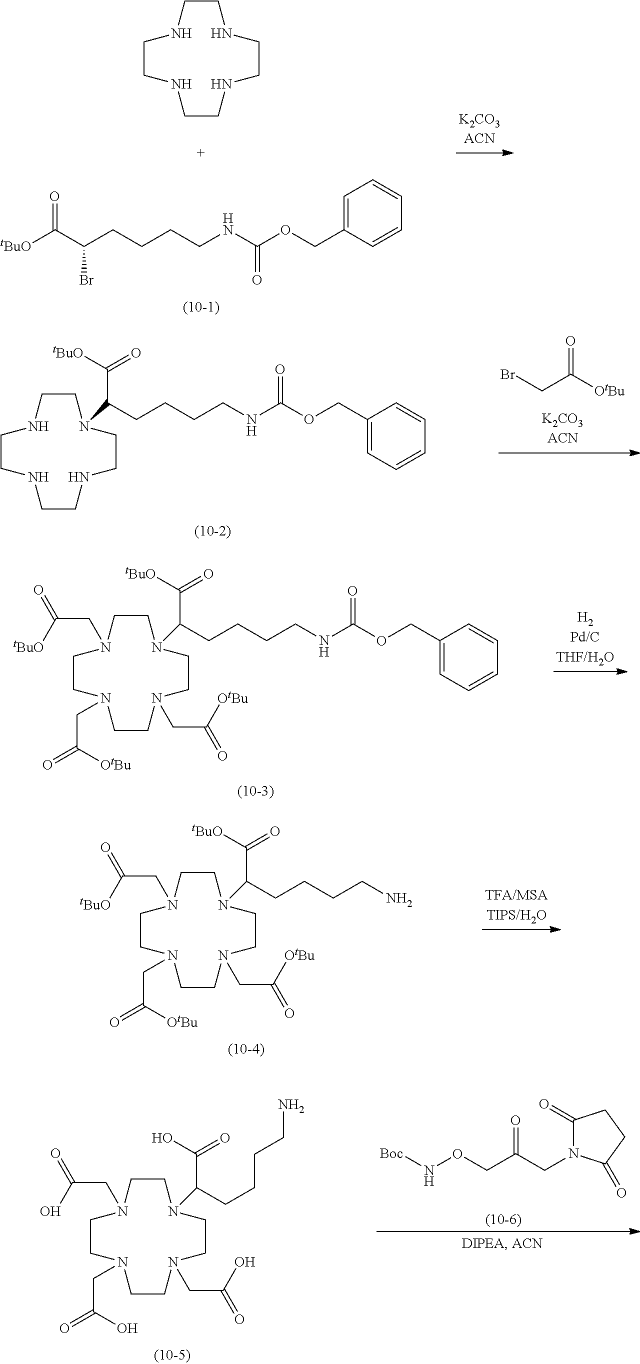

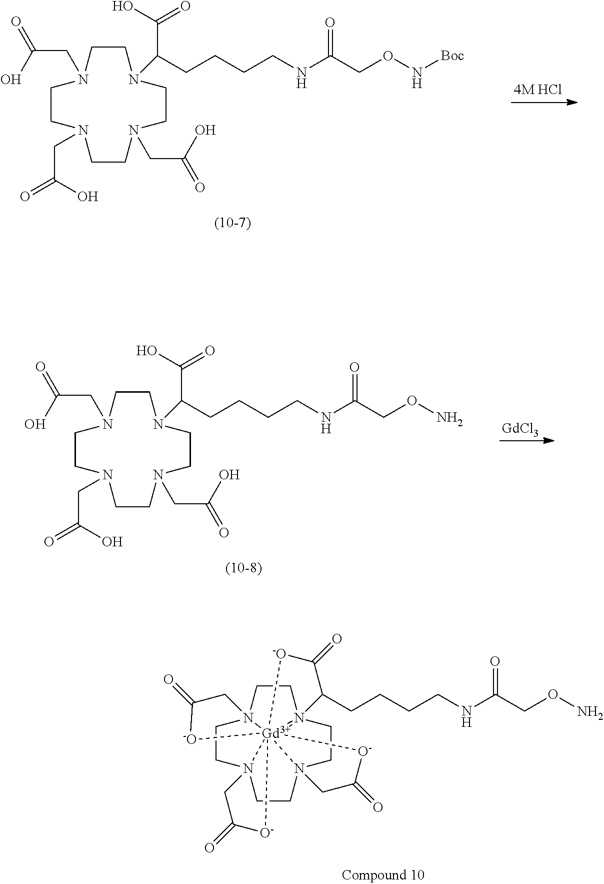

tert-Butyl-6-(((benzyloxy)carbonyl)amino)-2-(1,4,7,10-tetraazacyclododecan-1-yl)hexanoate (10-2)

Tetraazacyclododecane (0.842 g, 4.89 mmol) and triethylamine (1.136 mL, 8.13 mmol) were dissolved in acetonitrile (25 mL). To this solution was added tert-butyl 6-(((benzyloxy)carbonyl)amino)-2-bromohexanoate (10-1) (0.650 g, 1.63 mmol) and the starting material consumption followed over time by LC/MS. After 6 h the solvent was evaporated and the residue purified by preparative HPLC to yield 0.731 g (1.49 mmol, 91%) of white solid product: ¹H NMR (CDCl₃): δ 7.96 (br. s, 4H), 7.28 (m, 5H), 5.37 (br. s, 1H), 5.06 (s, 2H), 3.28-2.88 (m, 18H), 1.61 (m, 2H), 1.56-1.41 (m, 15H); ¹³C NMR (CDCl₃): δ 172.1, 156.7, 136.7, 128.5, 128.0, 127.6, 83.0, 66.4, 63.2, 47.0, 44.6, 43.3, 42.4, 40.4, 29.3, 28.4, 27.9, 24.1; LC/MS (ESI+): C₂₆H₄₅N₅O₄: m/z calcd 492.35 [MH⁺]; found 492.4 (MH⁺).

tri-tert-Butyl 2,2′,2″-(10-(6-(((benzyloxy)carbonyl)amino)-1-(tert-butoxy)-1-oxohexan-2-yl)-1,4,7,10-tetraazacyclododecane-1,4,7-triyl)triacetate (10-3)

tert-butyl 6-(((benzyloxy)carbonyl)amino)-2-(1,4,7,10-tetraazacyclododecan-1-yl)hexanoate (10-2) (0.955 g, 1.94 mmol) and potassium carbonate (2.685 g, 19.4 mmol) were dissolved in dry acetonitrile (20 mL). tert-butyl 2-bromoacetate (1.100 g, 5.64 mmol) dissolved in dry acetonitrile (40 mL) was added dropwise with starting material consumption followed by LC/MS over time. After 6 h the solvent was evaporated and the residue purified by preparative HPLC to yield 1.491 g (0.179mmol, 92%) of white solid product: LC/MS (ESI+): C₄₄H₇₅N₅O₁₀: m/z calcd 834.56 [MH⁺]; found 835.5 (MH⁺).

tri-tert-Butyl 2,2,,2″-(10-(6-amino-1-(tert-butoxy)-1-oxohexan-2-yl)-1,4,7,10-tetraazacyclododecane-1,4,7-triyl)triacetate (10-4)

Tri-tert-Butyl 2,2′,2″-(l0-(6-(((benzyloxy)carbonyl)amino)-1-(tert-butoxy)-1 -oxohexan-2-yl)-1,4,7,10-tetraazacyclododecane-1,4,7-triyl)triacetate (10-3) (1.200 g, 1.44 mmol) was added to a slurry of palladium on carbon (dry, 61.3 mg, 10% by mass) in anhydrous methanol (15 mL). The mixture was subject to two cycles of vacuum and hydrogen purge and then stirred under an atmosphere of hydrogen for 12 h. After evacuating the system of hydrogen, celite was added and the slurry filtered through a MeOH-wet bed of celite. The filtrate was concentrated in vacuo to a light yellow oil to yield 0.896 g (1.28 mmol, 89%) of product which was used in the next step without further purification: LC/MS (ESI+): C₃₆H₆₉N₅O₈: m/z calcd 700.52 [MH⁺]; found 700.7 (MH⁺).

2,2′,2″-(10-(5-Amino-1-carboxypentyl)-1,4,7,10-tetraazacyclododecane-1,4,7-triyl)triacetic acid (10-5)

Tri-tert-Butyl 2,2′,2″-(10-(6-amino-1-(tert-butoxy)-1-oxohexan-2-yl)-1,4,7,10-tetraazacyclododecane-1,4,7-triyl)triacetate (10-4) (0.896 g, 1.28 mmol) was dissolved in a mixture of TFA (15 mL), triisopropyl silane (900 μL) and water (900 μL) and the mixture was stirred at room temperature overnight. The volatiles were removed in vacuo to yield an oil 0.572 g (1.20 mmol, 94%) which was used in the next step without further purification: LC/MS (ESI+): C₂₀H₃₇N₅O₈: m/z calcd 476.27 [MH⁺]; found 476.5 (MH⁺).

2,2′,2″-(10-(14-Carboxy-2,2-dimethyl-4,8-dioxo-3,6-dioxa-5,9-diazatctradcean-14-yl)-1,4,7,10-tetraazacyciododecane-1,4,7-triyl)triacetic acid (10-7)

2,2′,2″-( 10-(5-Amino-1-carboxypentyl)-1,4,7,10-tetraazacyclododecane-1,4,7-triyl)triacetic acid (10-5) (0.572 g, 1.20 mmol) and diisopropylethylamine (1.05 mL, 1.26 mmol) were dissolved in dry DMF (10 mL). After 5 min, 2,5-dioxopyrrolidin-1-yl 2-(((tert-butoxycarbonyl)amino)oxy)acetate (10-6) (0.416 g, 1.44 mmol) was added and stirring continued for 24h. The solvent was evaporated and the residue purified by preparative HPLC to yield 0.652 g (1.00 mmol, 84%) of white solid product: ¹H NMR (d₆-DMSO): δ 4.15 (s, 2H), 3.79 (m, 4H), 3.61 (m, 2H), 3.56 (dd, 1H), 3.20-2.93 (m, 18H), 1.76 (m, 1H), 1.60 (m, 1H), 1.56-1.38 (m, 13H); LC/MS (ESI+): C₂₇H₄₈N₆O₁₂: m/z calcd 649.34 [MH⁺], found 649.6 (MH⁺).

2,2′,2″-(10-(5-(2-(aminooxy)acetamido)-1-carboxypentyl)-1,4,7,10-tetraazacyclododecane-1,4,7-triyl)triacetic acid (10-8)

2,2′,2″-(10-(14-carboxy-2,2-dimethyl-4,8-dioxo-3,6-dioxa-5,9-diazatetradecan-14-yl)-1,4,7,10-tetraazacyclododecane-1,4,7-triyl)triacetic acid (10-7) (50.0 mg, 77.1 μmol) was dissolved in 4M HCl in dioxane (4 mL) and stirred at room temperature overnight. The volatiles were removed in vacuo. The residue was redissolved in water and the pH adjusted to 7 using ammonium hydroxide. After lyophilisation, the solid was redissolved in water and subjected to a UV titration using arsenazo III to determine the concentration of ligand (23.5 mg, 42.4 μmol, 43 mM).

LC/MS (ESI+): C₂₂H₄₀N₆O₁₀: m/z calcd 549.29 [MH⁺]; found 549.3 (MH⁺) Compound 10

The stock solution of 2,2′,2″-(10-(5-(2-(aminooxy)acetamido)-1-carboxypentyl)-1,4,7,10-tetraazacyclododecane-1,4,7-triyl)triacetic acid (10-8) (23.5 mg, 42.4 μmol) was treated with GdCl₃.6H₂O (16.1 mg, 43.3 μmol) and the pH adjusted to 6.8. After 12 h of stirring Na₂H₂EDTA (0.79 mg, 2.12 μmol) was added and the solution stirred for a further 2h. The pH was adjusted to 7 and the solution purified by preparative HPLC to yield product (21.4 mg, 30.4 μmol, 72 %). Both Arsenazo III and xylenol orange test were negative demonstrating the absence of non-5 chelated Gd(III)

LC/MS (ESI+): C₂₂H₃₆GdN₆O₁₀: m/z calcd 703.18 [MH⁺]; found 703.3 (MH⁺)

### Example 5

### Preparation of NOT A Compounds 11-14

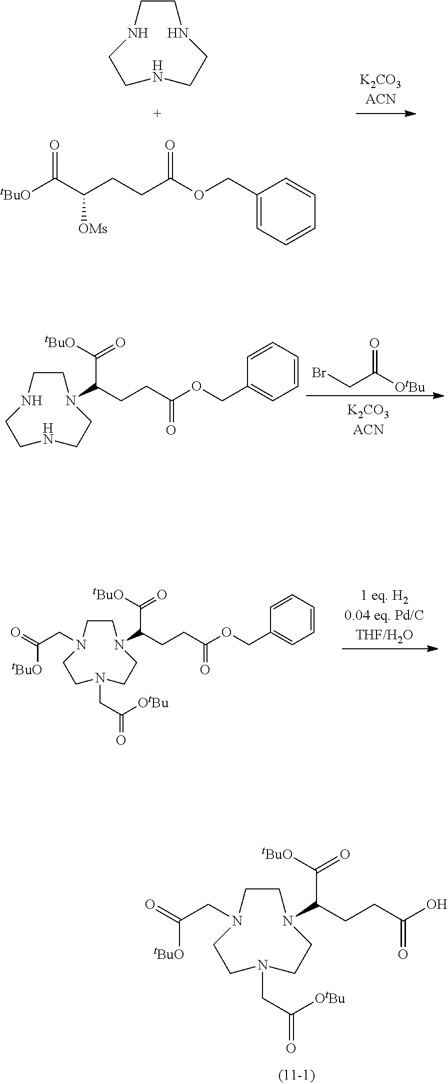

2-(R)-2-(4,7,10-tris tert-butylcarboxymethyl-1,4,7-triazacyclonon-1-yl)-pentanedioic acid-1-tert-butylester (11-1) was prepared following a literature procedure (Org. Process Res. Dev., 2009, 13, 535-542)).

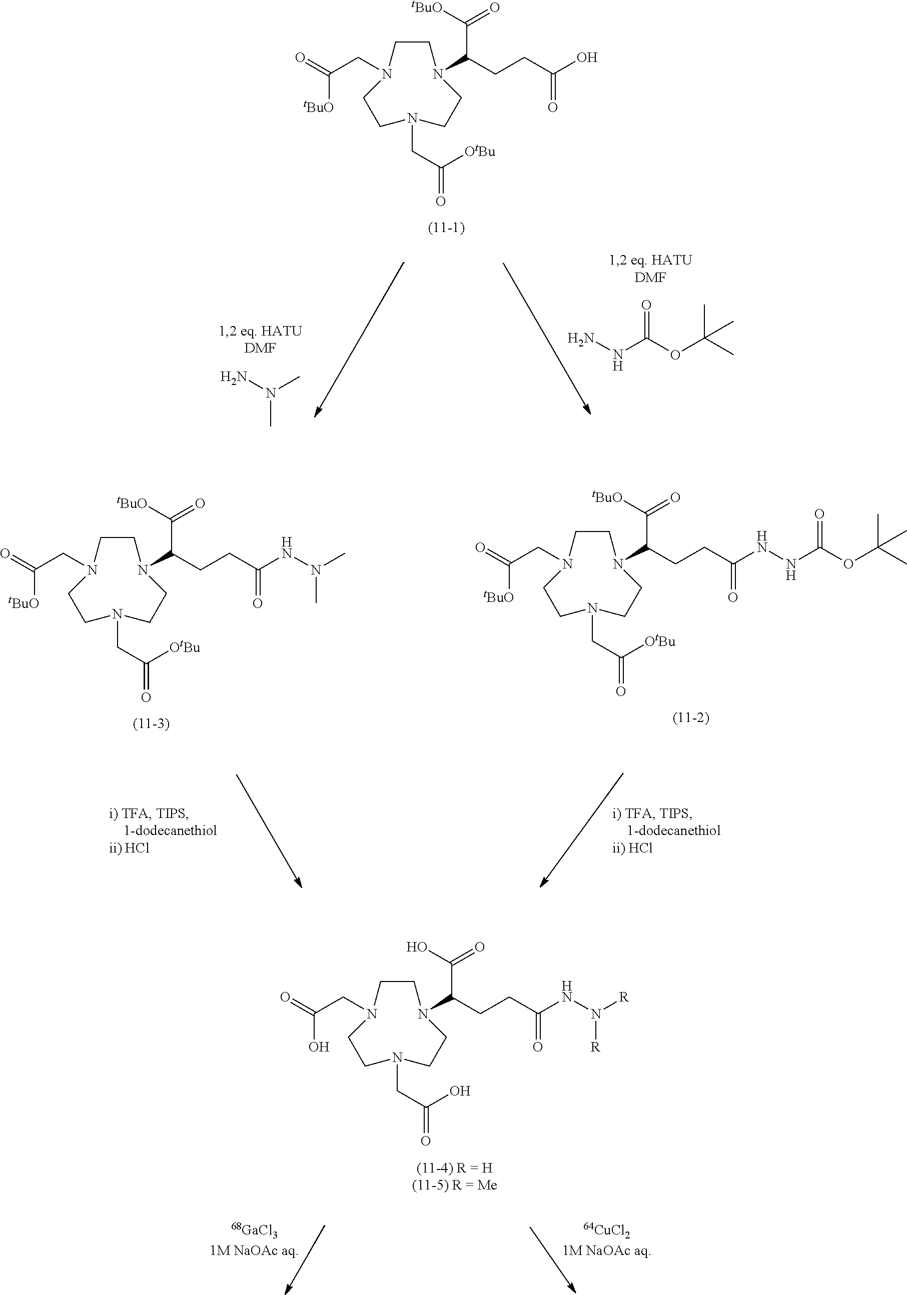

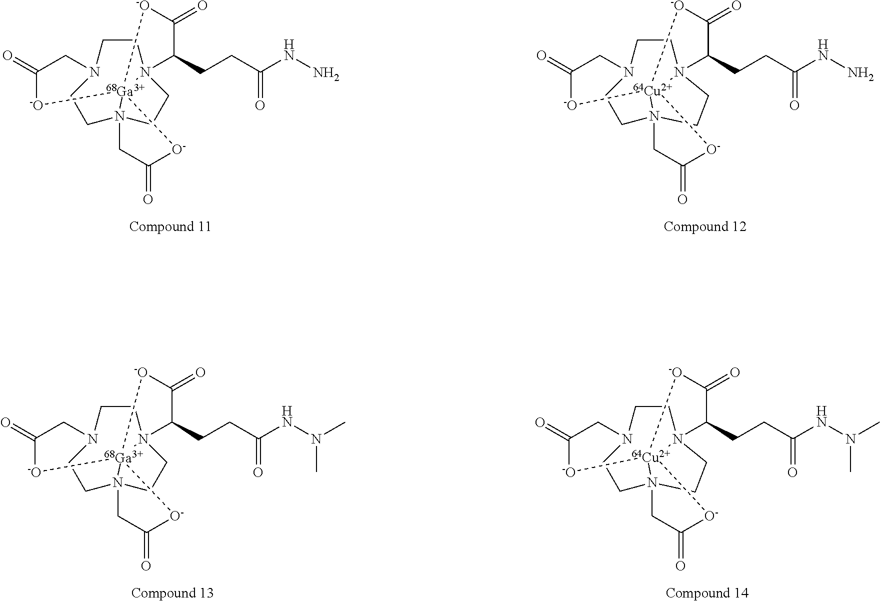

2-(R)-2-(4,7,10-tris tert-Butylcarboxymethyl-1,4,7-triazacyclonon-1-yl)-pentanedioic acid-1-N′-tert-butoxycarbonyl-N-hydrazide (11-2)

2-(R)-2-(4,7,10-tris tert-Butylcarboxymethyl-1,4,7-triazacyclonon-1-yl)-pentanedioic acid-1-tert-butylester (11-1) (152 mg, 280 μmol) and O-(7-azabenzotriazol-1 -yl)-N,N,N′ ,N′- tetraethyluronium hexafluorophosphate (HATU, 125.5 mg, 330 μmol) were dissolved in dry DMF (10 ml). After 5 min, solid tert-butyl-semicarbazide (43.6 mg, 330 μmol) was added and stirring continued for 24 h. The solvent was evaporated and the residue purified by reverse-phase preparative-HPLC to yield 39 mg (59 μmol, 21%) of the white solid product.

LC/MS (ESI+): C₃₂H₆₀N₅O₉: m/z calcd 658.44 [MH⁺]; found 658.4
2-(R)-2-(4,7,10-tris Carboxymethyl-1,4,7-triazacyclonon-1-yl)-pentanedioic acid-1-N′-tert-butoxycarbonyl-N-hydrazide (11-4)

2-(R)-2-(4,7,10-tris tert-Butylcarboxymethyl-1,4,7-triazacyclonon-1-yl)-pentanedioic acid-1-N′-tert-butoxycarbonyl-N-hydrazide (11-2) (52 mg, 79 μmol) was dissolved in a mixture of TFA (1.5 mL), triisopropyl silane (90 μL) and 1-dodecanethiol (90 μl) and the mixture was stirred at room temperature overnight. The volatiles were removed in vacuo and the residue redissolved in 6M HCl. After 3 h stirring at room temperature, the solution was lyophilized. The residue was redissolved in water and the pH adjusted to 7 using ammonium hydroxide.

¹H (d₆-DMSO): δ=4.41 (s, 4H), 4.14 (dd, 1H), 3.76-3.54 (m, 12H), 3.06 (t, 2H), 1.05 (ddt, 2H)
¹³C (d-DMSO): δ=175.5, 173.6, 172.4, 64.2, 56.0, 51.8, 50.1, 46.5, 30.5, 24.3. LC/MS (ESI+): C₁₅H₂₇N₅O₇: m/z calcd 390.20 [MH⁺]; found 390.1
Di-tert-Butyl 2,2′-(7-(tert-butoxy)-5-(2,2-dimethylhydrazinyl)-1,5-dioxopentan-2-yl)-1,4,7-triazonane-1,4-diyl)(S)-diacetate (11-3)

2-(R)-2-(4,7,10-tris tert-Butylcarboxymethyl-1,4,7-triazacyclonon-1-yl)-pentanedioic acid-1-tert-butylester (11-1) (152 mg, 280 prnol) and O(7-azabenzotriazol-1-yl)-N,N,N′,N′- tetraethyluronium hexafluorophosphate (HATU, 125.5 mg, 330 μmol) were dissolved in dry DMF (10 ml). After 5 min, N,N-dimethylhydrazine (19.8 mg, 330 μmol) was added and stirring continued for 24 h. The solvent was evaporated and the residue purified by reverse-phase preparative-HPLC to yield 0.123 g (0.21 mmol, 75%) of the white solid product. LC/MS (ESI+): C₂₉H₅₅N₅O₇: m/z calcd 586.42 [MH⁺]; found 586.6

(S)-2,2′-(7-(1-carboxy-4-(2,2-dimethylhydrazinyl)-4-oxobutyl)-1,4,7-triazonane-1,4-diyl)diacetic acid (11-5)

Di-tert-butyl 2,2′-(7-(1-(tert-butoxy)-5-(2,2-dimethylhydrazinyl)-1,5-dioxopentan-2-yl)-1,4,7-triazonane-1,4-diyl)(S)-diacetate (11-3) (80 mg, 0.137 mmol) was dissolved in a mixture of TFA (1.5 mL), triisopropyl silane (90 μL) and 1-dodecanethiol (90 μl) and the mixture was stirred at room temperature overnight. The volatiles were removed in vacuo and the residue redissolved in 6M HCl. After 3 h stirring at room temperature, the solution was lyophilized. The residue was redissolved in water and the pH adjusted to 7 using ammonium hydroxide. The solvent was lyophilized and the residue purified by reverse-phase preparative-HPLC to yield 22.0 mg (0.053 mmol, 38%) of the white solid product:

¹H (d₆-DMSO): δ=4.35 (s, 4H), 4.12 (dd, 1H), 3.75-3.52 (m, 18H), 3.04 (t, 2H), 2.64 (ddt, 2H)
LC/MS (ESI+): C₁₇H₃₂N₅O₇: m/z calcd 418.23 [MH⁺]; found 418.1

### ⁶⁴Cu-Labeled Conjugates:

1 mCi of ⁶⁴CuCl₂ in sodium acetate buffer (1 M, pH 4.5), was added into a vial containing 10 μg of Compound 11-4 and left to label at room temperature for 20 min. Radiochemical purity was assessed by RP-HPLC on a Restek Ultraaqueous C18 column (250 mm×3 mm×5 μm) under acidic conditions (Solvent A: H₂O+0.1% TFA, Solvent B: MeCN+0.1% TFA; 0-10 min, 0-20% B; 10-15min, 20-95% B; 15-17min, 95% B, 17-18min, 95-0% B; 18-20min, 0% B).

⁶⁴Cu-NODAGA-Hyd (Compound 12); Rₜ: 8.23 min (76%)

### ⁶⁸Ga-Labeled Conjugates:

A ⁶⁸Ge/⁶⁸Ga generator was eluted with 0.5 mL of HCl 6N. The eluate (15 mCi) was neutralized with 0.2 mL sodium acetate buffer (3M, pH 4.0) and added into a vial containing 10 μg of either Compound 11-4 or Compound 11-5. Both ligands were labeled at 60° C. for 15 min.

Radiochemical purity was assessed by RP-HPLC on a Restek Ultraaqueous C18 column (250mm×3mm×5 μm) under acidic conditions (Solvent A: H₂O+0.1% TFA, Solvent B: MeCN+0.1% TFA; 0-10 min, 0-20% B; 10-15 min, 20-95% B; 15-17 min, 95% B, 17-18 min, 95-0% B; 18-20 min, 0% B) ⁶⁸Ga-NODAGA-Hyd (Compound 11); Rₜ: 5.60 min (73%) ⁶⁸Ga-NODAGA-diMe (Compound 13); Rₜ: 8.57 min (100%).

### Example 6

### In Vitro Binding of Compounds with BSA

To demonstrate the selective binding of Compound 1 to aldehyde functionality in a biological setting, bovine serum albumin (BSA) with enhanced levels of aldehyde functionality was prepared and a comparison of the changes in Ti relaxivity after incubation with Compound 1 and Compound 2 were measured.

A solution of glutaraldehyde (100 μL, 25% wt solution in water) was added to a solution of bovine serum albumin (100 mg) dissolved in phosphate buffered saline (2 mL, pH 7.4, 0.25 mM) and left to stir at room temperature for 5 min. To this solution was added sodium cyanoborohydride (25 mg) and the solution stirred overnight at 4° C. A BSA protein standard without the addition of glutaraldehyde was run in parallel as a control. Both protein mixtures were purified on PD-10 Sephadex G25 desalting columns (GE Healthcare), eluted with water, to remove excess glutaraldehyde. Protein concentrations were assessed using the ‘BCA Protein Assay Kit’ (Thermo Scientific). The glutaraldehyde functionalized protein (BSA-ALD) had a concentration of 20 mg/mL, whilst the control protein (BSA) had a concentration of 18.4 mg/mL. The aldehyde concentration of each protein was estimated using a standard DNPH literature protocol. BSA-ALD had an aldehyde concentration of 16 nmol aldehyde/mg of protein, BSA had an aldehyde concentration on 1.2 nmol aldehyde/mg of protein

Aliquots of BSA-ALD (3 mg, 150 μL) or BSA (3mg, 163 μL) were treated for 24 h at 37° C. with either Compound 1 or Compound 2 at a range of concentrations (0.1-1.0 mM, concentrations equivalent to [Gd]:[aldehyde] ratio of 1:1, 2:1, 3:1, 4:1 and 5:1) with a total volume of 300 μl maintained for all samples. After 24 h longitudinal (T₁) relaxation measurements were recorded using a Bruker mq20 Minispec at 0.47 T and 37° C. Longitudinal (T₁) relaxation was acquired via an inversion recovery experiment on 10 inversions of duration ranging between 0.05×T₁ and 10×T₁. Relaxivity (r₁) was determined from the slope of a plot of 1/T₁ vs [Gd] for 5 concentrations of Gd(III).

After the measurements were complete sodium cyanoborohydride (10 mg) was added to each sample to reduce any hydrazone functionality and irreversibly bind the probe to the protein. After a further 2 h incubation at 37° C. longitudinal (T₁) relaxation measurmements for all samples were measured again.

Solutions of Compound 1 and Compound 2 (concentration range: 0.1 mM-1.0 mM, in water) were run in parallel without protein as standard controls.

Separation of the free and any BSA-bound Gd probes was achieved by ultrafiltration (5,000 Da cut-off PLCC cellulosic membrane). Following separation longitudinal (T₁) relaxation measurements of the protein and free solution fractions were measured, and quantification of Gd content in each fraction was determined using an Agilent 8800ICP-QQQ system.

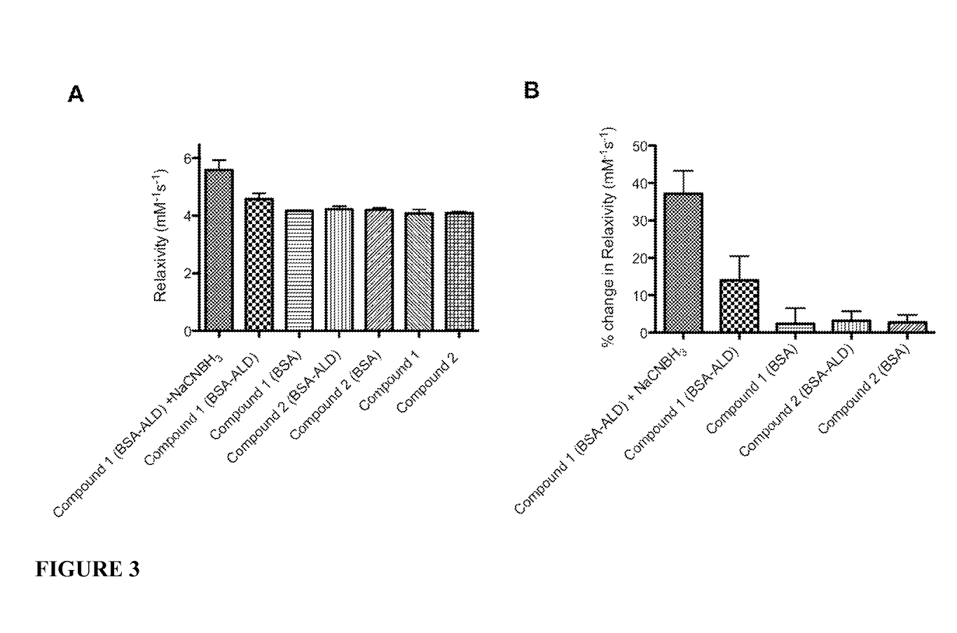

Relaxivity measurements for Compound 1 and Compound 2 with both BSA-ALD and BSA and their percentage change in relaxivity with respect to Compound 1and Compound 2 standards in solution (water) are given in FIGS. 3A and 3B respectively. No statistically significant difference was observed between the T₁ relaxivity measured for Compound 1 in solution (4.07 mM⁻¹s⁻¹, 310K, pH 7) and with BSA (4.17 mM⁻¹s⁻¹, 310K, pH 7). A statistically significant increase in T₁ relaxivity for Compound 1 incubated with BSA-ALD compared to Compound 1 in solution was observed (4.57 mM⁻¹s⁻¹, 310K, pH 7) and the increase showed greater statistical significance on the addition of the sodium cyanoborohydride reducing agent (5.59 mM⁻¹s⁻¹, 310K, pH 7). No statistically significant difference in relaxivity was seen for Compound 2 for either BSA (4.20 mM⁻¹s⁻¹, 310K, pH 7) or BSA-ALD (4.22 mM⁻¹s⁻¹, 310K, pH 7) samples with respect to the standard Compound 2 solution measurement (4.09 mm⁻¹s⁻¹, 310K, pH 7).

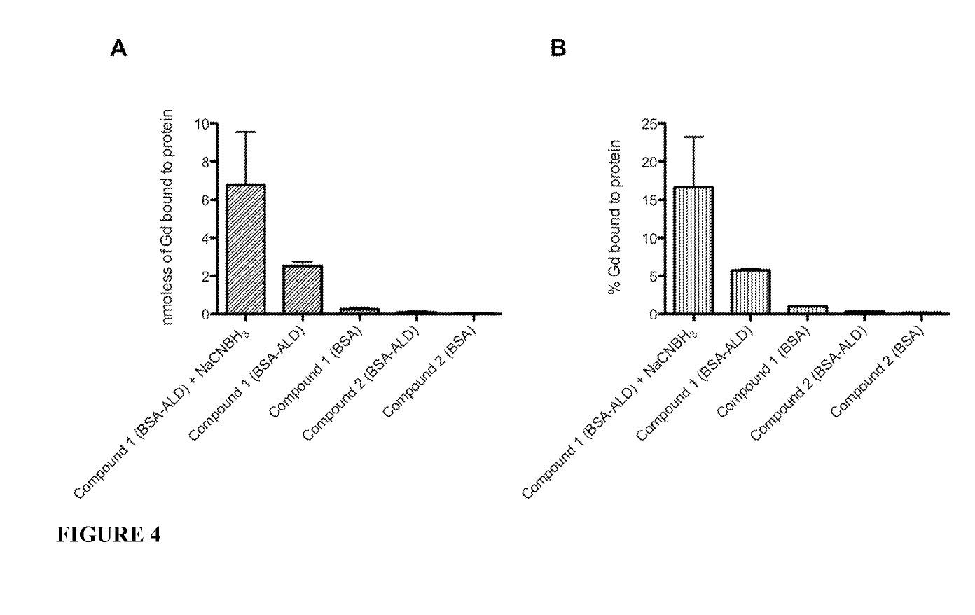

The amount of Gd associated with each protein fraction after separation from the free solution is shown in FIG. 4A and as a percentage of the total initial [Gd] concentration in FIG. 4B. No statistically significant amount of Gd was indicated to be bound to BSA or BSA-ALD for Compound 2. A ten fold increase in Compound 1 bound to protein was observed for BSA-ALD (2.52 nmol, 5.72% of total [Gd]) compared to BSA (0.24 nmol 0.58% of total [Gd]). The amount of Compound 1 bound to BSA-ALD increased to 6.78 nmol (16.63% of total [Gd]) on addition of reducing agent.

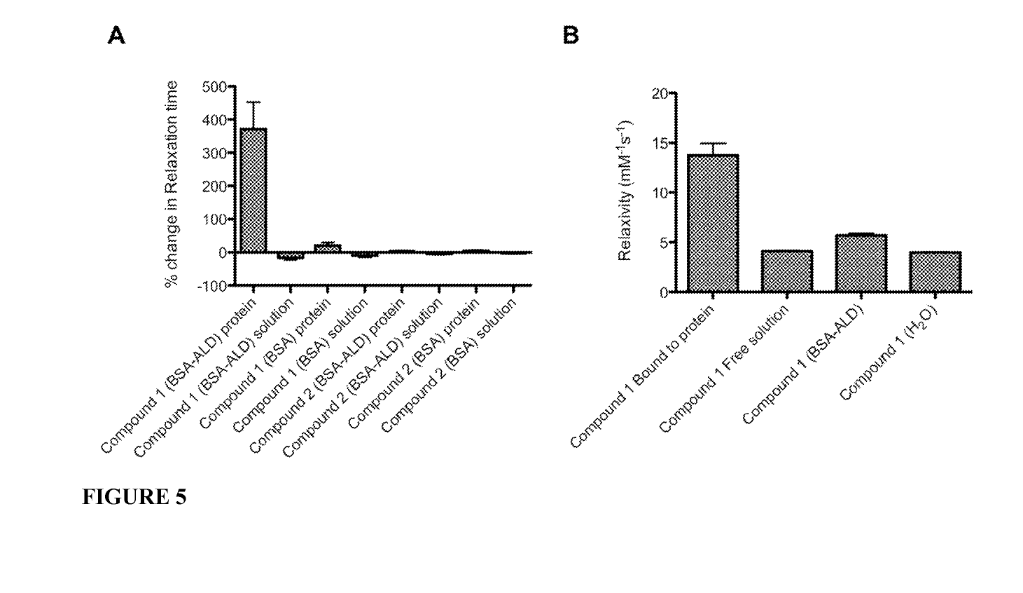

A comparison of longitudinal T₁ relaxation measurement for the protein bound and free solution fractions, showed that Compound 1 exhibited a significant increase in relaxation time for the protein fraction compared to the solution controls (FIG. 5A), and an associated decrease in free solution relaxation time, supporting an increased protein association. The Compound 2 relaxation times did not differ significantly from the standards for either BSA-ALD or BSA. Compound 1 incubated with BSA-ALD has a protein bound relaxivity of 13.74 mM⁻¹s⁻¹ (310K, pH 7) and free solution relaxivity of 4.02 mM⁻¹s⁻¹ (310K, pH 7) after separation, compared to a relaxivity of 4.09 mM⁻¹s⁻¹ (31 OK, pH 7) for Compound 1 in solution (FIG. 5B).

### Allysine Quantification

To correlate in vivo imaging data with allysine concentration, a HPLC analytical method was developed to quantify the amount of allysine present in lung tissue.

Lung tissue was hydrolysed in the presence of sodium 2-naphthol-6-sulfonate hydrate to form 2-amino-5-(1²,3²-dihydroxy-4,4,6,6-tetraoxido-5-oxa-4,6-dithia-1,3(1,6)-dinaphthalenacyclohexaphane-2-yl)pentanoic acid, a fluorescent derivative of allysine allowing detection and quantification by HPLC.

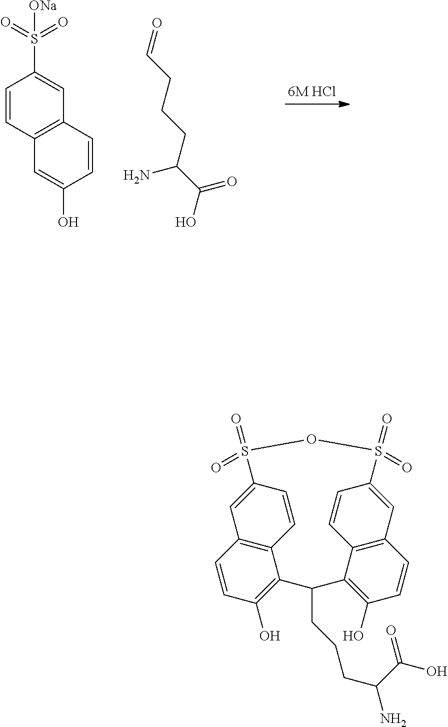

The lungs of bleomycin treated mice or control mice were hydrolysed for 24 h in a solution of 6M HCl (2 mL) containing sodium 2-naphthol-6-sulfonate hydrate (2% w/v), fluorescein (20 μL, 1 mM), sarcosine (100 μL, 4 mM) and hexanal (50 μL 8 mM). After heating at 110° C. for 24 h the solutions were cooled to room temperature and an aliquot (100 μL) neutralized with 6M NaOH (100 μL) and buffered with 0.6M borate buffer (100 μL, pH9) before analysis by HPLC.

### HPLC Method

Solvent A: 0.5M phosphate buffer with 0.2 mM EDTA and 1 mM MgCl₂, pH6.5, Solvent B: 60% Acetonitrile, 40% 0.5M Phosphate buffer containing 0.2 mM EDTA and 1 mM MgCl₂, pH6.5.

Method: 0-15min, 100-82.5% A; 15-18min, 82.5-50% A; 18-21min, 50-0% A; 21-28 min 0% A; 28-28.5 min, 0-100% A; 28.5-35 min 100% A.

Wavelength: 0-16 min, λₑₓ=285 nm, μₑₘ=313 nm; 16-20min, λₑₓ=460 nm, λₑₘ=515 nm; 20-35min λₑₓ=285 nm, λₑₘ=313 nm

Retention times: Peak areas for 2-amino-5-(1²,3²-dihydroxy-4,4,6,6-tetraoxido-5-oxa-4,6-dithia-1,3(1,6)-dinaphthalenacyclohexaphane-2-yl)pentanoic acid (retention time: 14.1 min) were corrected according to fluorescein and hexanal standards. The reaction of hexanal with sodium 2-naphthol-6-sulfonate hydrate to form 1²,3²-dihydroxy-2-pentyl-5-oxa-4,6-dithia-1,3(1,7)-dinaphthalenacyclohexaphane 4,4,6,6-tetraoxide was included as a reaction control (retention time: 26.9 min).

The hydroxyproline HPLC assay was performed on the same samples to quantify the amount of collagen present in each tissue sample to correlate allysine concentration with collagen concentration.

There was 2.7-fold more allysine observed in the lungs of bleomycin treated animals compared to control animals. This correlated with increased levels of collagen (91.7 μg/lung for bleomycin treated animal vs 54.1 μg/lung for control animals).

### Example 7

### Biodistribution of Compound 1

To test whether Compound 1 was substantially retained in the body after injection, normal A/3 mice (n=3) were injected intravenously with 100 μmol/kg of Compound 1. At 24 hours after injection the mice were euthanized, and tissues were removed, weighed, digested in nitric acid and analyzed for Gd content by ICP-MS. The percentage of the injected dose remaining in each tissue at 24 hours post-injection was as follows: blood (0.00015±0.00003), lung (0.17±0.08), heart (0.0052±0.0015), liver (0.31±0.09), spleen (0.029±0.009), stomach (0.0076±0.0026), intestine (0.0024±0.0003), kidney (0.092±0.018), muscle (0.068±0.014, assuming muscle is 40% of body mass). Together, the residual Gd in these tissues represents <0.7% of the injected dose indicating that Gd-Hyd is almost completely eliminated after intravenous administration.

### Example 8

### Magnetic Resonance (MR) Imaging in Mouse Models of Fibrosis Animal Models

Liver Fibrosis: Strain A/J male mice (Jackson Laboratories, Bar Harbor, Me.) were administered 0.1 mL of a 40% solution of CCl₄ (Sigma, St. Louis, Mo.) in olive oil by oral gavage three times a week for 18 weeks to induce fibrosis. Controls received only pure olive oil. Animals were imaged one week after the last injection to avoid acute effects of CCl₄.

Pulmonary Fibrosis: Pulmonary fibrosis was initiated in ten-week old male C₅₇/BL6 mice by transtracheal administration of bleomycin (BM, 2.5 U/kg) in PBS. Sham animals received only PBS.

### MR Imaging

### Liver Imaging (CCl₄ Mice)

Mice were imaged with T1-weighted imaging before and after bolus (tail vein) injection of probe (Compound 1 or Compound 2) using a 4.7T scanner. Image visualization and quantification was performed using the DICOM viewer Osirix. A region of interest (ROI) was placed over the entire liver section while avoiding major blood vessels. Axial slices that cover the entire liver is analyzed (>10 slices/mouse). The signal intensity of the muscle within each slice was also quantified by a separate ROI. To estimate noise, an ROI of the air outside the animal was measured and the standard deviation of this measurement was taken. The same analysis was performed on the pre and 30-min post injection images (3D FLASH sequence).

Contrast to noise ratio (CNR) is calculated using equation (1). SI=signal intensity, SD=standard deviation, and delta CNR is the absolute difference between the pre- and post- images (2).

CNR=(SIₗᵢᵥₑᵣ−SI_{muscle})/SDₐᵢᵣ (1)

Delta CNR={CNRₚₒₛₜ−CNRₚᵣₑ} (2)

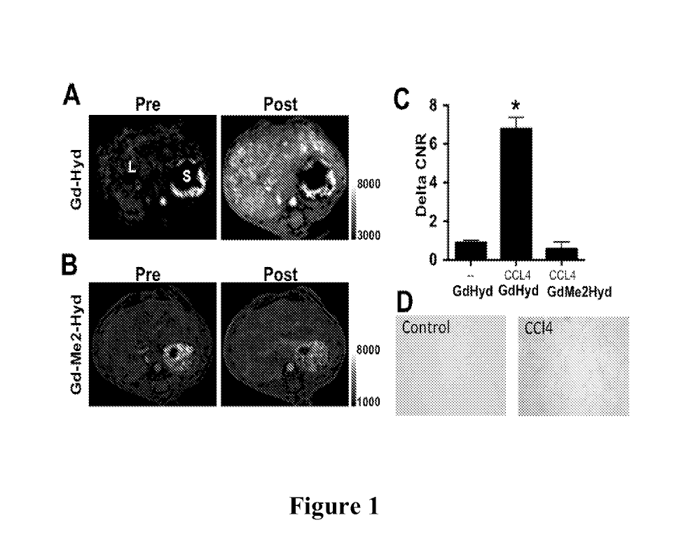

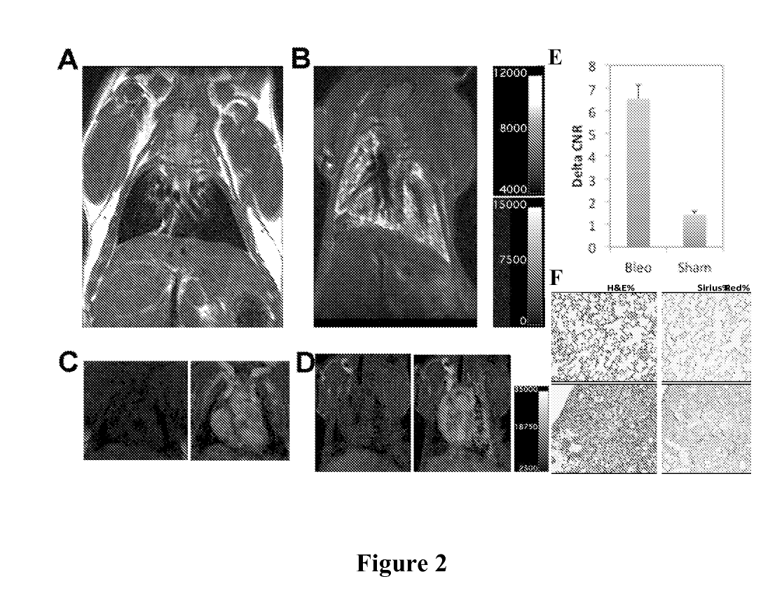

The results are show in FIGS. 1A-1D. FIG. 1A shows transaxial MR images before and after administration of Compound 1 to a CCL4-treated mouse (Ishak 5 fibrosis). The MR images post administration of Compound 1 exhibited strong enhancement in MR signal intensity in the liver. On the other hand, almost no enhancement was observed in age matched control mice with healthy livers. A control probe, Compound 2, which is a methylated version of Compound 1, exhibits similar pharmacokinetics, but does not bind to the peptidyl aldehydes in collagen. FIG. 2B shows that this methylated control, i.e., Compound 2, showed very little enhancement of the fibrotic liver. FIG. 2C shows the increase in MR contrast between the liver and skeletal muscle. A large and significant effect was seen only in fibrotic mice that received Compound 1, but not in control mice with healthy livers, nor in fibrotic mice that received the control probe Compound 2. FIG. 2D shows that Sirius Red staining confirmed that advanced fibrosis in fibrotic mice.

### Lung Imaging (Bleomycin Treated Mice)

Mice were imaged with T1-weighted imaging before and after bolus (tail vein) injection of probe (Compound 1 or Compound 2) using a 4.7T scanner. The images were gated for respiratory motion. The imaging protocol involved 1) multislice 2D rapid acquisition with refocused echo (RARE) imaging to delineate anatomy; 2) a baseline 3D ultrashort TE (UTE) sequence with respiratory gating; 3) a baseline 3D fast low angle shot (FLASH) angiography sequence; 4) bolus injection of 100 μmol/kg Compound 1; 5) the 3D FLASH sequence was repeated 5 times; 6) the 3D UTE sequence was repeated for 3 times. Images were analyzed using the program Osirix (www.osirix-viewer.com/). The post-injection 3D FLASH image was used to visualize the pulmonary vasculature. A region of interest (ROI) was manually placed over the lung tissue excluding major blood vessels. One ROI was placed over the left, while another was place over the right lobe of the lung. The ROIs were then copied exactly over to the UTE images to quantify probe intensity. Coronal slices that cover the entire lung were analyzed (>10 slices/mouse). The signal intensity of the muscle within each slice was also quantified by a separate ROI. This was done for pre- and post-probe UTE images. For each slice, the signal intensity (SI) in the lung and muscle was obtained. The standard deviation (SD) of the signal intensity in the air adjacent to the animal was used to estimate the noise. CNR and Delta CNR were calculated as in equations (1) and (2) above.

The results are shown in FIGS. 2A-2F. MR images of two mice were obtained: one administered bleomycin intratracheally 10 days prior to imaging in order to induce pulmonary fibrosis and a second mouse that was administered only phosphate buffered saline (sham) and which has normal lung architecture. These mice were imaged at baseline and then injected with Compound 1 and imaged further. FIGS. 2A and 2B show MR images of sham mouse and mouse with pulmonary fibrosis, respectively. FIGS. 2C and 2D are images taken before and immediately after injection of Compound 1 and demonstrate similar enhancement of the blood pool in both mice. However with time, Compound 1 is cleared by the normal mouse (FIG. 2A), but there is significant MR image signal enhancement remaining in the fibrotic mouse (FIG. 2B). The change in contrast between the lung tissue and adjacent skeletal muscle (CNR) were quantified. FIG. 2E shows the increase in CNR measured 1 hour after injection of Compound 1 for both mice. The contrast was 6-fold higher in the fibrotic mice. FIG. 2F shows that histology confirms the presence of fibrosis in the fibrotic mouse.

### Example 9

### Magnetic Resonance Imaging in Mice After 6 or 12 Weeks of CCl₄ Treatment

### Animal Model

Strain A/J male mice (Jackson Laboratories, Bar Harbor, Me.) were administered 0.1 ml of a 40% solution of CCl4 (Sigma, St. Louis, Mo.) in olive oil by oral gavage, three times a week for either 6 (n=14) or 12 weeks (n=10), to induce fibrosis at different stages. Controls received only pure olive oil (n=12).

Animals were imaged prior to and immediately following injection of the imaging probe. After imaging, the animals were sacrificed and the liver was removed for histopathological analyses.

Animals were anesthetized with isoflurane (1-2%) and placed in a specially designed cradle with body temperature maintained at 37° C. The tail vein was cannulated for intravenous (iv) delivery of the contrast agent while the animal was positioned in the scanner. Imaging was performed at 4.7T using a small bore animal scanner (Bruker Biospec) with a custom-built volume coil. The mouse was imaged prior to and following a bolus iv injection of Compound 1 (100 μmol/kg). The image sequence was a three dimensional fast low angle shot (3D FLASH) acquisition with repetition time (TR=15.3 ms), echo time (TE=1.54 ms), flip angle=15°, field of view 48×24×24 mm and matrix size 192×96×96 for a resolution of 250 μm isotropic and 25 used 4 averages.

### Image Analysis

Image analysis was performed using the Osirix software. A region of interest (ROI) was manually traced encompassing the liver parenchyma while avoiding major blood vessels. A second ROI was placed on the dorsal muscle visible in the same image slice to quantify the signal intensity in the muscle for comparison. Seven ROIs were placed in the field of view without any tissue (air) to measure the noise in the image. More than 20 axial slices per mouse across the entire liver was analyzed in this fashion. The same analysis was performed on the pre-injection and 15-minute post injection images.

To quantify signal enhancement in the liver, contrast to noise ratio (CNR) was calculated using equation 1 below. SI=signal intensity, SD=standard deviation. An average of all image slices was calculated for the pre-injection images (CNRpre) and for the post-injection images (CNRₚₒₛ). The liver enhancement for each mouse was expressed as ΔCNR, the difference between the pre-injection CNR and post-injection CNR (equation 2).

CNR=(SIₗᵢᵥₑᵣ−SI_{muscle})/SDₐᵢᵣ (1)

ΔCNR=CNRₚₒₛₜ−CNRₚᵣₑ (2)

Differences among groups were tested with repeated measures ANOVA, followed by Student-Newman-Keuls post hoc test with P<0.05 considered as significant.

### Tissue Analysis

Formalin-fixed samples were embedded in paraffin, cut into 5 μm-thick sections and stained with Sirius Red according to standard procedures. Sirius red-stained sections were analyzed using ImageJ (rsbweb.nih.gov/ij/) to quantify the percentage of the slide stained in red. The mRNA expression of LOX, LOXL1, and LOXL2 in liver tissue were quantified by real-time PCR using Taqman primers (Life Technologies, Grand Island, N.Y.). The Taqman primer sets were Mm00495386_m1 for LOX, Mm01145738_m1 for LOXL1 and Mm00804740_m1 for LOXL2. The 25 expression of each gene was normalized to the expression of the gene 18 s.

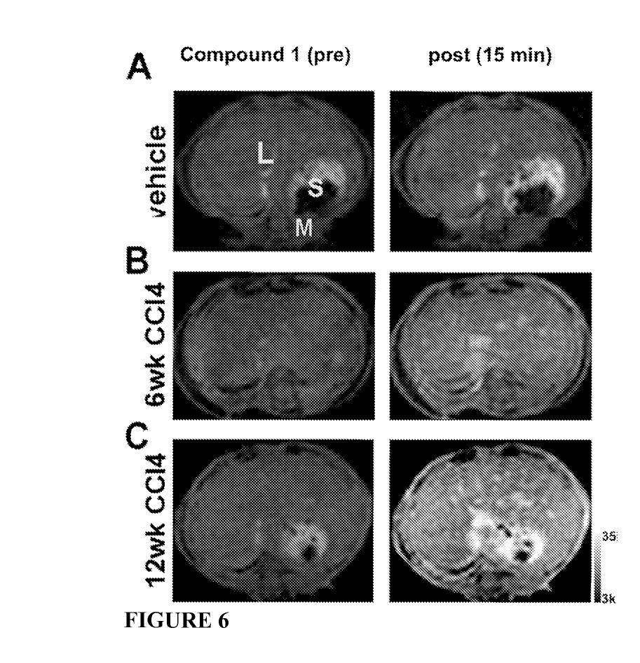

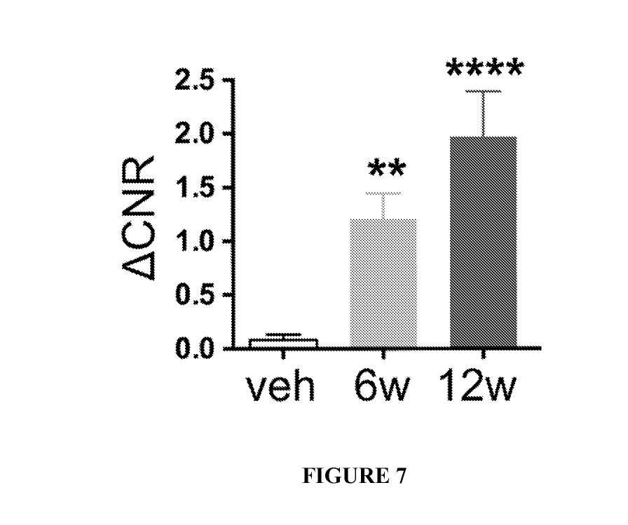

The results are shown in FIG. 6. In vehicle treated animals there is little signal enhancement in the liver at 15 min post injection, but there was marked enhancement over baseline images for the mice that received CCl₄ for either 6 or 12 weeks. This is shown in FIG. 6 where axial images are displayed pre- and post-Compound 1 injection. Pre-injection images showed similar contrast among vehicle (FIG. 6A, left panel), 6-week CCl₄ treatment (FIG. 6B, left) and 12-week CCl₄ treatment (FIG. 1C, left). Contrast enhancement seen in the post-Compound 1 injection image of 12-week CCl₄-treated animal (FIG. 6C, right panel) was not seen in the vehicle treated control (FIG. 6A, right). Enhancement was intermediate in the 6-week CCl₄-treated animal (FIG. 6B, right). The change in liver:muscle contrast-to-noise ratio, ΔCNR, increases from 0.1±0.2 in vehicle treated sham animals to 1.2±0.8 in 6-week CCl₄-treated animals (p<0.01, FIG. 7). ΔCNR further increases to 2.0±1.3 in the 12-week cohort (p<0.0001 compared to vehicle, FIG. 7). This is a 12-fold increase in Compound 1 induced ΔCNR in the 6-week CCl₄-treated animals and a 20-fold increase in the 12-week CCl₄-treated animals.

### Example 10

### Histology of 6-Week or 12-Week CCl₄-Treated Mice

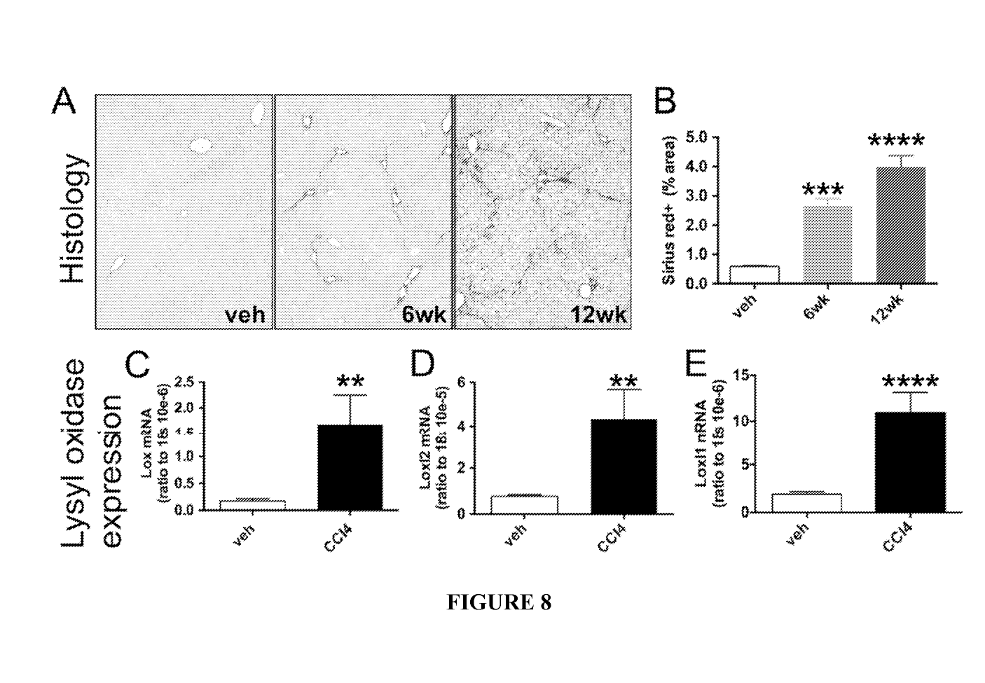

Increased Sirius Red staining was observed in the 6 week and 12 week CCU groups compared to the vehicle control. FIG. 8A (middle panel) shows diffuse fibrosis in the 6-week animals with extensive portal fibrosis but occasional bridging fibrosis (FIG. 8A, middle). Dense Sirius Red staining with complete bridging fibrosis was seen in the 12-week cohort (FIG. 8A, right). Quantitatively, Sirius Red staining increased from 0.6±0.2% in vehicle, to 2.7±0.8% in 6-week animals, to 4.0±1.2% in 12-week CCl₄ liver (FIG. 8B). Lysyl oxidase mRNA expression determined by qRT-PCR confirmed that in these animals, LOX (FIG. 8C), LOXL2 (FIG. 8D), and LOXL1 (FIG. 8E) gene expression were increased with CCU treatment.

### Example 11

### Compound 1 Imaging of Liver Fibrosis Regression

### Animal Model

Strain A/J male mice (Jackson Laboratories, Bar Harbor, Me.) were administered 0.1 ml of a 40% solution of CCl₄ (Sigma, St. Louis, Mo.) in olive oil by oral gavage, three times a week for 6 weeks and then allowed to recover for an additional 6 weeks (n=7). Animals were imaged prior to and immediately following injection of the imaging probe using the same protocol as in the previous example.

### Results

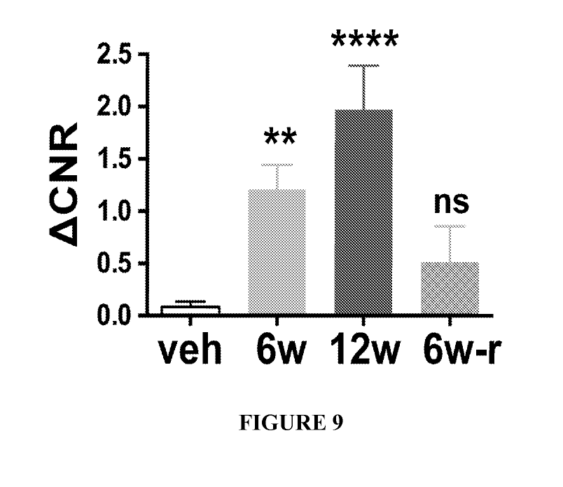

Compared to mice that were imaged after 6 weeks of CCl₄ treatment (6 w, FIG. 9), mice treated with CCl₄ for 6 weeks followed by a 6 week recovery period (6w-r, FIG. 9) showed a reduced ΔCNR from 1.2±0.8 in the 6-week CCl₄-treated only animals (p<0.01 compared to vehicle control) to 0.5±0.9 (not statistically significantly different from vehicle control). This is a 58% reduction in Compound 1 enhancement. Mice that continued to receive CCl₄ for 12 weeks showed higher ΔCNR. The imaging studies were consistent with histology. Sirius Red staining was diminished in the withdrawal group (1.4±0.4%) compared to mice that continued to receive CCl₄ (3.8±0.7%, P<0.00001), but higher than the vehicle control group (0.5±0.2%, P0.00001).

### Example 12

### Compound 2 Imaging

We compared Compound 2 to Compound 1 in terms of their ability to image fibrosis. Compound 2 has a very similar structure to Compound 1 but the hydrazide functional group has been dimethylated. The resulting dimethylhydrazide in Compound 2 is incapable of undergoing irreversible reaction with aldehyde moieties. Using the same animal model and imaging paradigm as described in the previous Example, mice that had been treated with CCl₄ for 12 weeks or mice that received vehicle for 12 weeks were imaged.

For mice imaged with Compound 2, slight enhancement of the liver was observed in vehicle treated (ΔCNR=0.6±0.9) and 12-week CCl₄-treated animals (ΔCNR=0.5±0.5), but there was no difference in ΔCNR between the vehicle treated and CCl₄-treated mice. However, for Compound 1, ΔCNR was 20-fold higher in the CCl₄-treated group (ΔCNR=2.0±1.3) compared to the vehicle group (ΔCNR=0.1±0.2).

### Example 13

### Compound 1 Imaging of Bleomycin-Induced Lung Fibrosis in Mice Cohorts

Animal model

Pulmonary fibrosis was initiated in 10-week-old male C₅₇/BL6 mice by transtracheal administration of bleomycin (Bleo; 2.5 U/kg) in PBS. Sham animals received only PBS. Animals were imaged prior to and immediately following injection of the imaging probe. After imaging, the animals were sacrificed and the lungs were removed for histopathological analyses. 3 cohorts were imaged: 1) vehicle only (n=16), 2) 1 week after Bleo (n=18), 3) 2 weeks after Bleo (n=12).

Animals were anesthetized with isoflurane (1-2%) and placed in a specially designed cradle with body temperature maintained at 37 ° C. The tail vein was cannulated for intravenous (iv) delivery of the contrast agent while the animal was positioned in the scanner. Imaging was performed at 4.7T using a small bore animal scanner (Bruker Biospec) with a custom-built volume coil. The mouse was imaged prior to and following a bolus iv injection of Gd-Hyd (100 μmol/kg). Two imaging sequences were used: a three dimensional fast low angle shot (3D FLASH) acquisition with repetition time (TR=15.3 ms), echo time (TE=1.54 ms), flip angle (FA=40°), field of view 48×24×24 mm and matrix size 192×96×96 for a resolution of 250 μm isotropic, 1 average; and a three dimensional ultrashort time to echo (3D UTE) acquisition with TR/TE/FA=8.0 ms/0.02 ms/40°, field of view 48×24×24 mm and matrix size 192×96×96 for a resolution of 250 μm isotropic, 1 average.

### Image Analysis

Image analysis was performed using the Osirix software. ROIs were manually traced on the right and left lung parenchyma while avoiding major blood vessels, on the right and left shoulder muscle, and 7 ROIs were placed in the field of view without any tissue (air) to measure the noise in the image. Coronal slices that cover the entire lung were analyzed (>10 slices per mouse). The same analysis was performed on the pre-injection and 30-minute post injection images.

To quantify signal enhancement in the lung, contrast to noise ratio (CNR) was calculated using equation 1 below. SI=signal intensity, SD=standard deviation. An average of all image slices was calculated for the pre-injection images (CNRₚᵣₑ) and for the post-injection images (CNRₚₒₛ). The lung enhancement for each mouse was expressed as ΔCNR, the difference between the pre-injection CNR and post-injection CNR (equation 2).

CNR=(SIₗᵢᵥₑᵣ−SI_{muscle})/SDₐᵢᵣ (1)

ΔCNR=CNRₚₒₛₜ−CNRₚᵣₑ (2)

Differences among groups were tested with repeated measures ANOVA, followed by Student-Newman-Keuls post hoc test with P less than 0.05 considered as significant.

### Tissue Analysis

Formalin-fixed samples were embedded in paraffin, cut into 5 μm-thick sections and stained with Sirius Red and with Hematoxylin and Eosin (H&E) according to standard procedures. Sirius red-stained sections were analyzed using ImageJ (rsbweb.nih.gov/ij/) to quantify the percentage of the slide stained in red. Slides were also analyzed by a pathologist and scored using the Ashcroft scale and the extent of lung injury was also assessed.

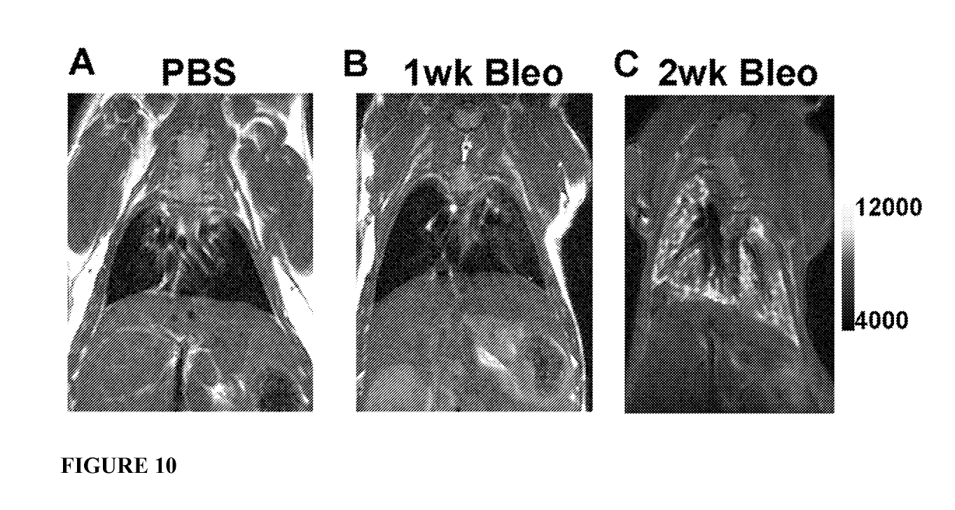

In PBS sham animals there is minimal signal enhancement in the lung at 30 min post injection, but there was marked enhancement over baseline images for the mice treated with bleomycin when imaged at either 7 or 14 days post bleomycin instillation. This is shown in FIG. 10 where coronal anatomical images are displayed in greyscale with the signal enhancement from Compound 1 is overlaid in false color. ΔCNR increased from 0.8±1.1 in the PBS sham animals, to 2.5±1.5(p<0.05) in the 1-week Bleo animals, and further to 4.3±1.3 (p<0.001) in the 2-week Bleo cohort (FIG. 10).

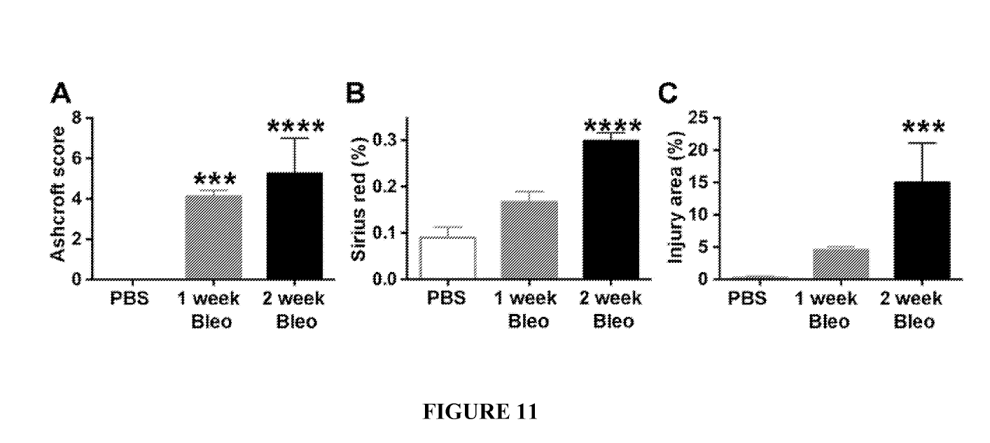

Ex vivo tissue analysis confirmed disease progression in mice 14 days after bleomycin compared to 7 days after bleomycin instillation. The average Ashcroft score, a measure of fibrosis severity, was 4.1±0.9 in the 1-week bleo animals, 5.3±3.5 in the 2-week bleo animals, and 0 in the PBS sham animals (FIG. 11A). The fraction of lung tissue staining positive with Sirius Red staining area was 0.09±0.06% in sham, 0.17±0.07% in 1-week bleo, and 0.30±0.04% in 2-week bleo cohort (FIG. 11B). The injury area increased from 0.3±0.7% (sham), to 4.6±1.3% (1-week Bleo), and further to 15.0±12.3% in 2-week Bleo animals (FIG. 11C). All three pathological measures confirmed fibrosis progression from sham to 1-week post bleo instillation animals, and further in the 2-week animals.

### Example 14

### Bleomycin Treatment Timing

The bleomycin model is known to create significant fibrosis that peaks at about 2 weeks post instillation of bleomycin. At later timepoints the mice begin to recover. A C_{57B}16 mouse treated with transtracheal instillation of bleomycin (2.5 u/kg) was imaged at 2 weeks and at 4 weeks after bleomycin treatment. Using the same imaging protocol as in the previous Example, it was found that ΔCNR was 2.3 at 2 weeks post bleomycin but this decreased to 0.9 at 4 weeks post bleomycin, a 61% reduction in Gd-Hyd enhancement.

Other embodiments are within the scope of the following claims.

1. A compound of formula (I): 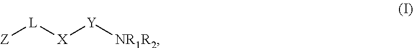
or a pharmaceutically acceptable salt thereof, wherein
X is —C(RₐR_{b})—, —C(S)—, or —C(O)—, in which each of Rₐ and R_{b}, independently, is H, alkyl, alkenyl, alkynyl, cycloalkyl, heteroaryl, or aryl;
Y is —N(R_{c})— or —O—, in which R_{c}is H, alkyl, alkenyl, alkynyl, or aryl;
L is —(CR_{d}Rₑ)ₙ—, —NH(CR_{f}R_{g})ₙ—, or —(CRₕRᵢ)ₙ—aryl-, in which each of R_{d}, Rₑ, R_{f}, R_{g}, Rₕ, and Rᵢ is independently in each instance H, alkyl, alkenyl, or alkynyl, and n is 1, 2, or 3;
Z is a chelate group comprising a metal ion and a first complexing group, the first complexing group forming a metal complex with the metal ion; and
each of R₁ and R₂, independently, is H or C₁-C₁₀ alkyl.

2. The compound of claim 1, wherein the first complexing group is a DOTA, NOTA, DO3AX, DO3AP, DOTP, DO2A2P, NOTP, NO2AP, NO2PA, TETA, TE2P, TE2A, TE1A1P, CBTE2P, CBTE1A1P, SBTE2A, SBTE1A1P, DTTP, CHX-A″-DTPA, Desferal, HBED, PyDO3P, PyDO2AP, PyDO3A, DIAMSAR, EDTA, DTP A, CB-TE2A, SarAr, PCTA, pycup, DEDPA, OCTAPA, AAZTA, DOTAIa, CyPic3 A, TRAP, NOPO, or CDTA moiety.

3. The compound of claim 1, wherein the metal ion is selected from Gd³⁺, Mn³⁺, Mn²⁺, Fe³⁺, Ce³⁺, Pr³⁺, Nd³⁺, Eu³⁺, Eu²⁺, Tb³⁺, Dy³⁺, Er³⁺, Ho³⁺, Tm³⁺, Yb³⁺, and Cr³⁺, or is an ion of a radioisotope selected from the group consisting of ⁶⁷Ga, ⁶⁸Ga, Al-¹⁸F, 64Cu, ¹¹¹In, ⁵²Mn, ⁸⁹Zr, ⁸⁶Y, ²⁰¹Tl, ^{94m}Tc, and ^{99m}Tc.

4. The compound of claim 1, wherein X is —C(RₐR_{b})—, —C(S)—, or —C(O)—, in which each of Rₐ and R_{b}, independently, is H, C₁-C₁₀ alkyl, C₂-C₁₀ alkenyl, C₂-C₁₀ alkynyl, or aryl.

5. (canceled)

6. The compound of claim 1, wherein Y is —N(R_{c})— or —O—, in which R_{c}is H, C₁-C₁₀ alkyl, C₂-C₁₀ alkenyl, C₂-C₁₀ alkynyl, or aryl.

7. (canceled)

8. The compound of claim 1, wherein L is —(CH₂)ₙ—, —NH(CH₂)ₙ—, or —(CH₂)ₙ—aryl-, in which n is 1, 2, or 3.

9. (canceled)

10. The compound of claim 1, wherein each of Rₐ and R_{b}, independently, is H or CH₃.

11. The compound of claim 1, wherein the compound is 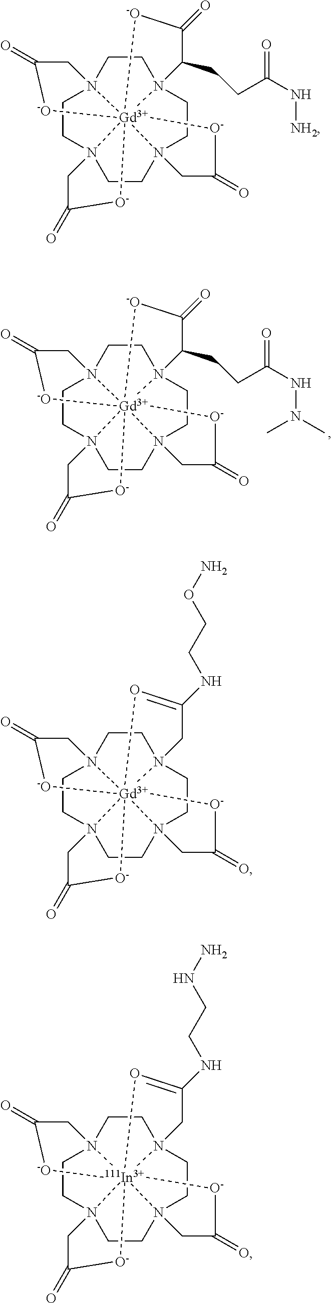 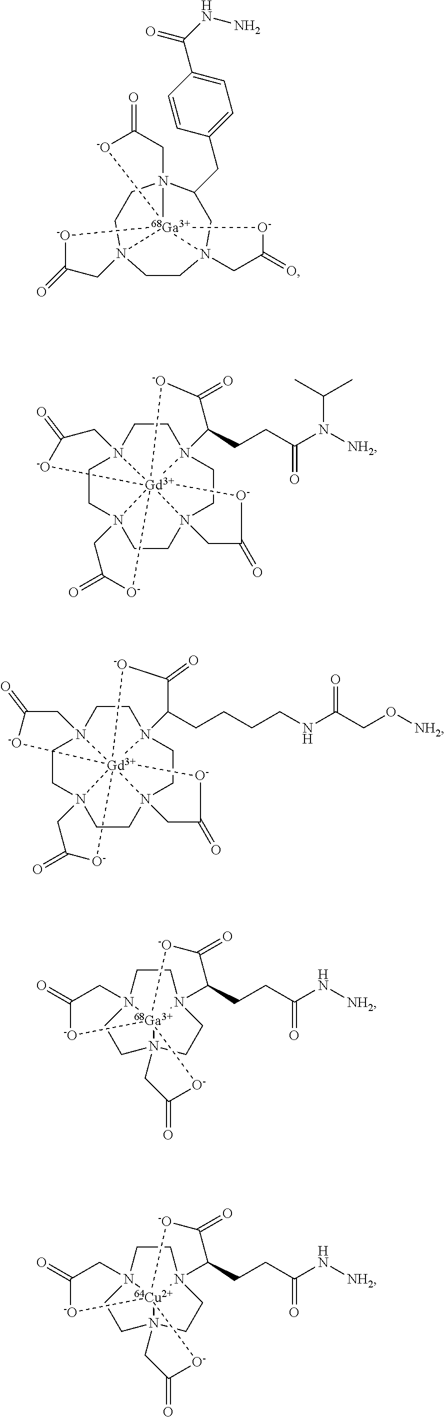 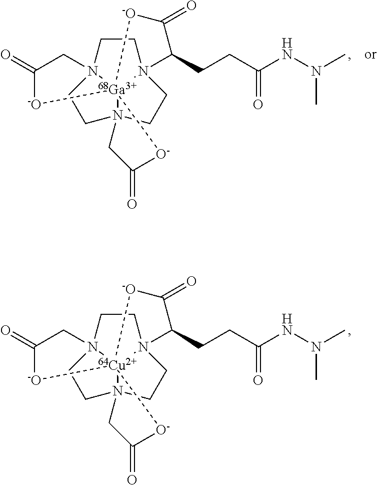
pharmaceutically acceptable salt thereof.

12. The compound of claim 1, wherein Z further comprises a water molecule complexed with the metal ion.

13. The compound of claim 12, wherein the compound is 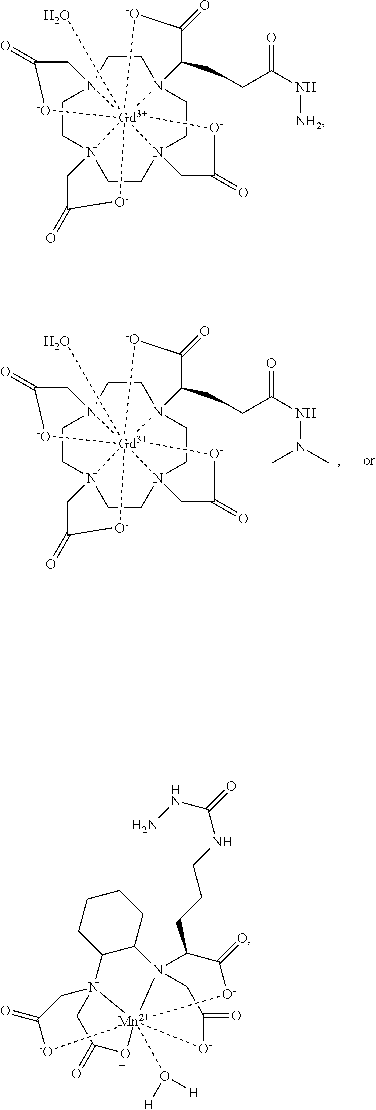
or a pharmaceutically acceptable salt thereof.

14. (canceled)

15. A method for assessing lysyl oxidase activity in an extracellular matrix of a biological sample or in a tissue or tumor in a mammal comprising administering to the extracellular matrix or to the mammal an imaging agent comprising a —NR—NH₂ or —O—NH₂ group, wherein R is H, C₁-C₁₀ alkyl, C₂-C₁₀ alkenyl, C₂-C₁₀ alkynyl, or aryl; and acquiring an image of the extracellular matrix, tissue, or tumor after administration of the imaging agent.

16. (canceled)

17. (canceled)

18. A method for imaging an extracellular matrix of a biological sample or a tissue or tumor in a mammal comprising:
administering to the extracellular matrix or to the mammal an imaging agent comprising a —NR—NH₂ or —O—NH₂ group, wherein R is H, C₁-C₁₀ alkyl, C₂-C₁₀ alkenyl, C₂-C₁₀ alkynyl, or aryl; and acquiring an image of the extracellular matrix, tissue, or tumor after administration of the compound.

19. (canceled)

20. (canceled)

21. A method for assessing the level of fibrosis in a tissue of a mammal, comprising administering to the mammal an imaging agent comprising a —NR—NH₂ or —O—NH₂group, wherein R is H, C₁-C₁₀ alkyl, C₂-C₁₀ alkenyl, C₂-C₁₀ alkynyl, or aryl; and acquiring an image of the mammal after administration of the imaging agent.

22. A method for diagnosing a fibrotic disease in a mammal, comprising administering to the mammal an imaging agent comprising a —NR—NH₂ or —O—NH₂ group, wherein R is H, C₁-C₁₀ alkyl, C₂-C₁₀ alkenyl, C₂-C₁₀ alkynyl, or aryl; and acquiring an image of the mammal after administration of the imaging agent.

23. The method of claim 22, wherein the fibrotic disease is selected from the group consisting of: pulmonary fibrosis, chronic obstructive pulmonary disease, pulmonary arterial hypertension, heart failure, hypertrophic cardiomyopathy, myocardial infarction, atrial fibrillation, diabetic nephropathy, systemic lupus erythematosus, polycystic kidney disease, glomerulonephritis, end stage renal disease, nonalcoholic steatohepatitis, alcoholic steatohepatitis, hepatitis C virus infection, hepatitis B virus infection, primary sclerosing cholangitis, inflammatory bowel disease, scleroderma, atherosclerosis, glaucoma, diabetic retinopathy, radiation induced fibrosis, surgical adhesions, cystic fibrosis, idiopathic pulmonary fibrosis, and cancer.

24.-35. (canceled)
